SEQUENCE LISTING (12 sequences)

SEQ ID NO: 1 - DNA, 6046 bp, synthetic construct
  misc_feature 1..6046: note = sh(FABP4/5) plasmid
  source 1..6046: mol_type = other DNA
cctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtaaatcaacgggactttccaaaatgtcgtaacaactccgccccattgacgcaaatgggcggtaggcgtgtacggtgggaggtctatataagcagagctcgtttagtgaaccgtcagatcagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagccaccatggtttctaagggagaagaactctttactggtgttgtcccaattctggttgagctggatggtgatgtgaatggccacaaattctctgtgtctggtgaaggtgaaggagatgcaacttatggaaagctgactctgaagttcatttgtacaacaggaaagctgccagtgccttggccaactctggtgaccaccctgacttatggtgttcaatgtttcagcagataccctgaccacatgaagcagcatgacttctttaaatctgcaatgccagaaggttatgttcaggagaggacaatcttctttaaggatgatggaaattataagacaagggcagaagtgaagtttgaaggtgatacactggttaacagaattgagctgaaaggcattgattttaaggaagatggaaacattctgggtcacaagctggagtacaactataattctcacaatgtttacattatggcagataagcagaggaatggaattaaggctaatttcaagattagacacaacattgaggatggatctgtccaactggcagaccattaccagcagaacacccctattggtgatggcccagttctcctcccagataatcactatctcagcactcaatctgctctgtccaaagaccctaatgagaaaagagaccacatggtcctcctggagtttgtgacagcagcaggaattactctgggaatggatgagctgtacaagggtaagtcactgactgtctatgcctgggaaagggtgggcaggagatggggcagtgcaggaaaagtggcactatgaacccactagtttgacaattaatcataagcatagtataatacaactcactatagcaattgtactaaccttcttctctttcctctcctgacaggaggagccatcatggccaaactcacttctgcagtcccagtcctcacagcaagggatgttgcaggggctgtagagttctggactgacagattaggattctccagagactttgttgaagatgattttgctggtgttgtcagagatgatgtcaccctcttcatctcagcagttcaggaccaagttgtccctgacaacacccttgcttgggtctgggtcagaggcctagatgagctttatgcagaatggtcagaagtagtcagcacaaatttcagggatgcctctggcccagccatgacagaaattggtgaacaaccttggggaagggaatttgccctcagagaccctgctggaaattgtgtccattttgtagctgaggaacaggactaaagctagaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttaattaacgttctgcagtatttagcatgccccacccatctgcaaggcattctggatagtgtcaaaacagctggaaatcaagtctgtttatctcaaactttagcattttgggaataaatgatatttgctatgctggttaaattagattttagttaaatttcctgctgaagctctagtatgataagtaacttgacctaagtgtaaagttgagatttccttcaggtttatatagtccctatcagtgatagagacctcggtcttcacctgaggtttttcaaaagtagttgacaattaatcatcggcatagtatatcggcatagtataatacgactcactataggagggccaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaataatagctagaggaagactttttggaaaagattaaaaacccgcttcggcgggtttttttatgcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgttcttaatcgatactagtgctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctgaggtttttcaaaagtagttgacaattaatcatcggcatagtatatcggcatagtataatacgactcactataggagggccaccatggaccctgttgtgctgcaaaggagagactgggagaaccctggagtgacccagctcaacagactggctgcccaccctccctttgcctcttggaggaactctgaggaagccaggacagacaggcccagccagcagctcaggtctctcaatggagagtggaggtttgcctggttccctgcccctgaagctgtgcctgagtcttggctggagtgtgacctcccagaggctgacactgtgtaaccctaagcttctagacttaattaa

SEQ ID NO: 2 - protein, 18 aa, synthetic construct
  REGION 1..18: note = ATS-9R
CKGGRAKDRRRRRRRRRC

SEQ ID NO: 3 - DNA, 22 bp, synthetic construct
  misc_feature 1..22: note = FABP4 Forward primer
  source 1..22: mol_type = other DNA
tgaaatcaccgcagacgacagg

SEQ ID NO: 4 - DNA, 23 bp, synthetic construct
  misc_feature 1..23: note = FABP4 Reverse primer
  source 1..23: mol_type = other DNA
gcttgtcaccatctcgttttctc

SEQ ID NO: 5 - DNA, 21 bp, synthetic construct
  misc_feature 1..21: note = FABP5 Forward primer
  source 1..21: mol_type = other DNA
tggtttacccaggatcattcc

SEQ ID NO: 6 - DNA, 22 bp, synthetic construct
  misc_feature 1..22: note = FABP5 Reverse primer
  source 1..22: mol_type = other DNA
cctgaagaataccagagagctt

SEQ ID NO: 7 - DNA, 21 bp, synthetic construct
  misc_feature 1..21: note = Leptin Forward primer
  source 1..21: mol_type = other DNA
tgagtttgtccaagatggacc

SEQ ID NO: 8 - DNA, 18 bp, synthetic construct
  misc_feature 1..18: note = Leptin Reverse primer
  source 1..18: mol_type = other DNA
gccatccaggctctctgg

SEQ ID NO: 9 - DNA, 22 bp, synthetic construct
  misc_feature 1..22: note = adiponectin Forward primer
  source 1..22: mol_type = other DNA
caatgtacccattcgctttact

SEQ ID NO: 10 - DNA, 20 bp, synthetic construct
  misc_feature 1..20: note = adiponectin Reverse primer
  source 1..20: mol_type = other DNA
catacacctggagccagact

SEQ ID NO: 11 - DNA, 23 bp, synthetic construct
  source 1..23: mol_type = other DNA
catcactgccacccagaagactg

SEQ ID NO: 12 - DNA, 23 bp, synthetic construct
  source 1..23: mol_type = other DNA
atgccagtgagcttcccgttcag

(19) United States
(12) Patent Application Publication
(10) Pub. No.: US 2025/0387323 A1
(43) Pub. Date: Dec. 25, 2025

(54) MICROSTRUCTURE FOR PREVENTION AND TREATMENT OF OBESITY AND OBESITY-DERIVED TYPE 2 DIABETES, COMPRISING COMPLEX OF GENE AND ADIPOCYTE-TARGETING GENE CARRIER

(71) Applicant: IUCF-HYU (INDUSTRY-UNIVERSITY COOPERATION FOUNDATION HANYANG UNIVERSITY), Seoul (KR)

(72) Inventors: Yong Hee KIM, Seoul (KR); Shayan FAKHRAEI LAHIJI, Seoul (KR); Hee Kyung CHOI, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 18/880,915

(22) PCT Filed: Jul. 4, 2023
(86) PCT No.: PCT/KR2023/009447

§ 371 Date: Jan. 3, 2025

(30) Foreign Application Priority Data
Jul. 4, 2022 (KR) 10-2022-0081838
Jul. 4, 2023 (KR) 10-2023-0086716

(51) Int. Cl. A61K 9/00; A61K 38/10; A61K 47/69; A61P 3/04; C12N 15/113

(52) U.S. Cl. CPC A61K 9/0021; A61K 38/10; A61K 47/6949; A61P 3/04; C12N 15/113; C12N 2310/531

(57) ABSTRACT

The present invention relates to a microstructure for the prevention and treatment of obesity and obesity-derived type 2 diabetes, comprising a complex of a gene and an adipocyte-targeting gene carrier. Being capable of suppressing the expression of FABP4 and FABP5 with the gene and the adipocyte-targeting gene carrier, the present invention can exhibit excellent prophylactic and therapeutic effects on obesity and obesity-derived type 2 diabetes.

### TECHNICAL FIELD

The present invention relates to a microstructure for prevention and treatment obesity and obesity-derived type 2 diabetes, including a complex of a gene and an adipocyte-targeting gene carrier.

### BACKGROUND ART

Existing obesity treatments are focused on fat absorption and appetite suppression, so they have side effects such as fatty stools and mental illness. Even drugs that have been developed to overcome these side effects also have limitations in that weight is restored immediately when the medication is stopped. In addition, existing obesity treatments are injections, so they have the disadvantage of causing trypanophobia, as well as the low stability of the drug during long-term treatment and the need to visit the hospital every time.

Administration routes for delivering drugs to the body include oral, injection, and transdermal administrations. Oral administration is a convenient way to increase patient compliance, and the active ingredient can be delivered to the body in the form of capsules, tablets, and syrup. However, the active ingredient may be deactivated due to first-pass metabolism in the liver, and it has been reported that the absorption rate of biopharmaceuticals is actually low. Injection administration is used to accurately and quickly exert the efficacy of drugs and therapeutic agents, which are administered to the body by penetrating the skin barrier. Although the activity of the active ingredient delivered by injection is well maintained, there are disadvantages such as the risk of infection, inaccurate dose, trypanophobia, and pain.

To overcome the limitations of existing oral and injection administration routes, various microstructure-based transdermal drug delivery systems have been developed, including minimally invasive microneedles. Microstructures are mainly manufactured in biodegradable (dissolving), solid, coated, and hollow forms. Biodegradable microstructures are transdermal delivery systems that are formulated with various substances, including polymers and active pharmaceutical ingredients (APIs/cosmetics or pharmaceuticals), into microneedle forms, and then inserted into the skin to be dissolved by body fluids to deliver the loaded substances painlessly.

Fatty acid-binding protein 4 (FABP4) and fatty acid-binding protein 5 (FABP5), which are targeted in the present invention, are positioned in adipose tissues, and when FABP4 is lost, FABP5 replaces FABP4. This is a factor related to fatty acid absorption and fat droplet size, and the expression of FABP4 and FABP5, which are involved in fat droplet size and fatty acid uptake and storage, increases in an obesity model. In addition, increased insulin resistance may lead to obesity-derived type 2 diabetes.

The present invention employs an RNAi system that inhibits gene expression of FABP4 and FABP5. Using a plasmid-based short hairpin RNA, sh(FABP4/5), the present invention aims at treating obesity and obesity-derived type 2 diabetes. A non-viral gene/carrier complex was developed using a prohibitin-binding protein (PBP)-9R peptide, which specifically binds to the prohibitin receptor overexpressed in the nuclear membrane and cell membrane of differentiated adipocytes.

In addition, existing gene-based adipocyte-targeting obesity treatments were developed as injections, and thus, there is a high possibility that patients will discontinue treatment due to the pain from the injection needle, patients' resistance, and the need for injection application education. Therefore, in the present invention, a gene-based adipocyte-targeting obesity treatment was loaded onto a minimally invasive microstructure that overcomes the problems of existing injections, thereby demonstrating its efficacy.

Existing obesity treatment drugs are focused on fat absorption and appetite suppression, but the present invention can contribute to the fundamental prevention and treatment of obesity and obesity-derived type 2 diabetes by inhibiting genes related to fat accumulation with an adipocyte-targeting non-viral gene/carrier. Existing injectable obesity treatments have low therapeutic effects and satisfaction due to the pain and the resistance to injection, but the present invention aims to maximize patients' application convenience and thus increase therapeutic effects by employing a microstructure, which is a minimally invasive platform.

### DISCLOSURE

### Technical Problem

One aspect is to provide a microstructure for preventing or treating obesity or obesity-derived type 2 diabetes, including a complex including an obesity or obesity-derived type 2 diabetes treatment gene and an adipocyte-targeting carrier.

Another aspect is to provide a patch for preventing or treating obesity or obesity-derived type 2 diabetes, including the microstructure.

Still another aspect is to provide a method of manufacturing a microstructure for preventing or treating obesity or obesity-derived type 2 diabetes, the method including: (a) a step of preparing an obesity or obesity-derived type 2 diabetes treatment gene; (b) a step of preparing an adipocyte-targeting carrier; and (c) a step of manufacturing a microstructure including a complex including the gene of Step (a) and the carrier of Step (b).

### Technical Solution

The present invention provides a microstructure for preventing or treating obesity or obesity-derived type 2 diabetes, including a complex including an obesity or obesity-derived type 2 diabetes treatment gene and an adipocyte-targeting carrier.

The term “obesity” used herein does not simply refer to being overweight, but to a state in which body fat is excessively accumulated. This means that even a person who appears to be of normal weight on the outside may be considered obese when the body fat percentage is high. The body mass index (BMI) is usually used to determine obesity. Those with the BMI of 23 to 24.9 are determined to be overweight, those with the BMI of 25 to 29.9 are determined to be mildly obese, those with the BMI of 30 to 34.9 are determined to be moderately obese, and those with the BMI of 35 or more are determined to be severely obese. Obesity is caused by a combination of factors rather than a single cause, including wrong eating habits, including the Westernized dietary habits, decreased physical activity level, emotional factors, and genetic factors. Obesity caused in this way ultimately increases the risk of developing diseases such as fatty liver, type 2 diabetes, hyperlipidemia, cardiovascular diseases, and arteriosclerosis.

The term “diabetes” as used herein refers to a chronic disease characterized by relative or absolute deficiency of insulin, which results in glucose intolerance. The term “diabetes” of the present invention includes all types of diabetes, for example, type 1 diabetes, type 2 diabetes, and hereditary diabetes. Type 1 diabetes is insulin-dependent diabetes, which is mainly caused by destruction of β-cells. Type 2 diabetes is non-insulin-dependent diabetes, which is caused by insufficient insulin secretion after a meal or caused by insulin resistance.

In one embodiment of the present invention, the diabetes may be type 2 diabetes, and specifically, obesity-derived type 2 diabetes.

In one embodiment of the present invention, the obesity or obesity-derived type 2 diabetes treatment gene may target fatty acid-binding protein 4 (FABP4) or fatty acid-binding protein 5 (FABP5).

In one embodiment of the present invention, the obesity or obesity-derived type 2 diabetes treatment gene may be used without limitation in the form of a compound, nucleic acid, peptide, peptide mimic, substrate analog, aptamer, antibody, virus, or vector containing the nucleic acid, which can inhibit the activity of FABP4 (aP2) or FABP5.

In one specific example of the present invention, the obesity or obesity-derived type 2 diabetes treatment gene may be one or more selected from the group consisting of siRNA or shRNA that binds to an mRNA of FABP4 or FABP5 gene, or an antisense oligonucleotide, RNAi, siRNA, miRNA, shRNA, and ribozyme that reduce the expression of FABP4 or FABP5 protein.

In one embodiment of the present invention, an shRNA form may be used to utilize the PBP characteristics of binding to prohibitin that is overexpressed in the nuclear membrane and cell membrane of differentiated adipocytes and to achieve fundamental and long-term treatment of obesity.

In one specific example of the present invention, the sh(FABP4/5) gene that targets and inhibits FABP4 (aP2) and FABP5 may be used for the prevention and treatment of obesity or obesity-derived type 2 diabetes.

Since these genes themselves exhibit a negative charge due to the phosphate structure, it is not easy for the genes themselves to penetrate the cell membrane which exhibits a negative charge, due to electrical repulsion. Therefore, the genes must react with a positively charged substance to form a complex so that the overall charge is positive to more easily enter the cell, thereby improving gene expression within the cell. A substance that facilitates the delivery of genes into cells in this way is referred to as a gene carrier. A gene carrier refers to a substance that combines with a gene to help the delivery of the gene for improved delivery and high expression of the gene, and such gene carriers are mainly positively charged substances, and a gene/carrier complex is formed through the electrical interaction between the negatively charged gene and the positively charged gene carrier.

In one embodiment of the present invention, the carrier may be a non-toxic, non-viral peptide carrier. By using this, the action of gene decomposition enzymes in the body may be blocked to improve delivery efficiency and reduce off-target effects. In one specific example, an adipocyte-targeting carrier may be a PBP-9R peptide, specifically, as represented by SEQ ID NO: 2.

In one embodiment of the present invention, a complex including the gene and the carrier may be formed by including the obesity or obesity-derived type 2 diabetes treatment gene and the adipocyte-targeting carrier in a weight ratio of 1:1, 1:2, 1:3, 1:4, 1:5, 1:6, 1:7, 1:8, 1:9, or 1:10. When the obesity or obesity-derived type 2 diabetes treatment gene and the adipocyte-targeting carrier are included in the above-described weight ratio, a complex may be stably formed.

In one embodiment of the present invention, the size of the complex including the gene and the carrier may be 100 to 1,000 nm, 100 to 800 nm, 100 to 600 nm, 100 to 500 nm, or 200 to 400 nm.

In one embodiment of the present invention, the microstructure may be in the form of a microneedle, and in one specific example, it may be an interlocking microstructure (interlocking microneedles, LMNs).

In one embodiment of the present invention, the microstructure may further include a biocompatible polymer. In one specific example, the biocompatible polymer may be a water-soluble polymer such as hyaluronic acid (HA), sodium carboxymethyl cellulose (Na-CMC), vinylpyrrolidone-vinylacetate copolymer, polyvinyl alcohol, and polyvinyl pyrrolidone, a sugar such as xylose, sucrose, maltose, lactose, and trehalose; or a mixture thereof.

In one specific example of the present invention, the biocompatible polymer may be HA.

In one embodiment of the present invention, the microstructure prepared by mixing a complex including the gene and the carrier and a biocompatible polymer may be used for the prevention and treatment of obesity or obesity-derived type 2 diabetes.

In one specific example of the present invention, the microstructure includes a biocompatible polymer that is dissolved in vivo, so that the substance contained in the microstructure can be gradually released in the skin, thereby maintaining the effect for a long time.

In one embodiment of the present invention, the microstructure may be soluble within the skin. In one specific example, the material forming the microstructure may be dissolved within the body so that the complex of the gene and carrier included in the microstructure may be effectively released into the skin.

In one embodiment of the present invention, the microstructure may further include a plasticizer, a surfactant, a preservative, an anti-inflammatory agent, and the like, in addition to the above-described components forming the microstructure.

As the plasticizer, for example, polyols such as ethylene glycol, propylene glycol, dipropylene glycol, butylene glycol, and glycerin may be used alone or in combination.

The length of the microneedle according to the present invention is not limited to a specific size.

In the present invention, the complex including the obesity or obesity-derived type 2 diabetes treatment gene and the adipocyte-targeting carrier may be included in an amount of 1.4% to 87% by weight, 2.7% to 65% by weight, or 5.4% to 43% by weight based on the total weight of the microstructure. When the complex is included in an amount less than 1.4% by weight based on the total weight of the microstructure, it may not exhibit a valid effect, and when it is included in an amount exceeding 87% by weight, the physical properties and durability of the microstructure may decrease.

The term “prevention” used in the present invention refers to any action that suppresses or delays the onset of a disease by the microstructure according to the present invention.

The term “treatment” used in the present invention refers to any action that ameliorates or beneficially changes the symptoms of a disease by the microstructure according to the present invention.

In addition, the present invention provides a patch for preventing or treating obesity or obesity-derived type 2 diabetes, including the above-described microstructure.

In the present invention, the patch may refer to a sheet having one or more of the microstructures attached thereto and having a surface with the attached microstructures manufactured so that the surface may be attached to the skin. The size of the sheet is not limited to a specific size, and may be appropriately adjusted according to the amount or attachment site of the complex including the obesity or obesity-derived type 2 diabetes treatment gene and the adipocyte-targeting carrier. In addition, one or more, preferably a plurality of microneedles may be attached to the surface of the sheet that may be attached to the skin.

In one embodiment of the present invention, the patch may include 1 to 10¹⁰⁰, 1 to 10⁵⁰, 1 to 10²⁰, or 1 to 10¹⁰ microneedles.

In one embodiment of the present invention, the patch may include microstructures with an aspect ratio of 500:1, 250:1, 200:1, 150:1, 100:1, 5:1, 1:1, 1:5, or 1:10.

In one embodiment of the present invention, the patch may include a base film and a plurality of microneedles as a microstructure. In one specific example, the patch may include a base film and microneedles in a ratio of 500:1, 250:1, 200:1, 150:1, 100:1, 5:1, 1:1, 1:5 or 1:10.

In one embodiment of the present invention, the patch may be used for preventing or treating obesity or obesity-derived type 2 diabetes, and may directly deliver a complex including a gene and a carrier to adipocytes, thereby increasing delivery efficiency.

In one embodiment of the present invention, the patch may be for topical application to the skin.

In addition, the present invention provides a method of manufacturing a microstructure for preventing or treating obesity or obesity-derived type 2 diabetes, the method including: (a) a step of preparing an obesity or obesity-derived type 2 diabetes treatment gene;
(b) a step of preparing an adipocyte-targeting carrier; and
(c) a step of manufacturing a microstructure including a complex including the gene of Step (a) and the carrier of Step (b).

In one embodiment of the present invention, Step (c) may consist of (i) a step of filling a mold with a material forming a microstructure including a complex including an obesity or obesity-derived type 2 diabetes treatment gene and an adipocyte-targeting carrier and mixing the material; and (ii) a step of drying and separating the mold.

In one embodiment of the present invention, the microstructure may be manufactured by a micromolding technique. In one specific example, the microstructure may form a soluble interlocking microstructure.

However, the above-described method of manufacturing the microstructure is an example, and any method of manufacturing the microstructure that may be used in the research field of the present invention may be used without limitation.

### Advantageous Effects

Obesity and obesity-derived type 2 diabetes can be treated by inhibiting the expression of FABP4 and FABP5 using the microstructure of the present invention, and long-term expression can be induced using shRNA (sh(FABP4/5)) among RNAi techniques.

In addition, since the efficacy is limited due to the short half-life and low targeting ability of the gene alone, an adipocyte-targeting peptide was used to penetrate the cell membrane and nuclear membrane, thereby improving the shRNA stability and helping the penetration and expression thereof, and to selectively enhance the adipocyte-targeting effect by binding to the prohibitin receptor on the surface of adipocytes.

In addition, it was confirmed that there was no change in gene efficacy in differentiated adipocytes due to hyaluronic acid (HA) used to produce the soluble microstructure of the present invention.

When the microstructure of the present invention was compared with the existing injection, it was confirmed that long-term stability was maintained at room temperature for eight weeks including the treatment period.

In addition, when a microstructure manufactured by mixing self-assembled oligopeptoplex (SA-OP) and HA was attached to an obese mouse model, it was confirmed that the targeting effect on visceral fat was maintained for 72 hours.

### DESCRIPTION OF DRAWINGS

FIG. 1A shows the results of confirming the stability of the sh(FABP4/5) gene and the prohibitin-binding protein (PBP)-9R carrier by electrophoresis. They were prepared in a weight ratio of 1:3 and measured at different time points in an acidic solution. FIG. 1B shows the results of confirming the stability of the sh(FABP4/5) gene and the PBP-9R carrier in terms of nanoparticle size. They were prepared in a weight ratio of 1:3 and measured at different time points in an acidic solution using dynamic light scattering (DLS).

FIG. 2 shows the results of confirming the stability of the sh(FABP4/5) gene and the PBP-9R carrier in serum by electrophoresis (+ at the top indicates the presence or absence of shRNA, + at the bottom indicates the presence or absence of the carrier, and incubation time (h) indicates the mouse serum incubation time).

FIG. 3 shows the formation of a soluble interlocking microstructure of the sh(FABP4/5) gene and the PBP-9R carrier using the micromolding technique. Thereafter, the stability of the gene in the complex was confirmed by electrophoresis using proteinase K (HA: hyaluronic acid; MN: microstructure).

FIG. 4A shows the results of confirming the properties of the soluble interlocking microstructure using a microscope. FIG. 4B shows a scanning electron microscope (SEM) image of the microstructure of the present invention.

FIG. 5A shows the results of microscopic confirmation after the application of the microstructure to mouse skin and the results of confirming the penetration thickness through hematoxylin and eosin (H&E) staining. FIG. 5B shows the results of measuring the breaking force to express the strength of the microstructure as numerical physical properties (HA-LMN: soluble interlocking microstructure made only of HA). FIG. 5C shows the results of dissolving HA (LMN) and SA-OP (LMN). The soluble interlocking microstructure was completely dissolved one hour after the application to the mouse.

FIG. 6A shows the results of transdermal water loss analysis of C57BL/6 mouse skin after LMN application. FIG. 6B shows the results of confirming the gene release amount at different time points by applying a soluble interlocking microstructure loaded with an SA-OP complex to pig skin in an environment similar to body temperature.

FIG. 7 shows the results of verifying the binding ability of the microstructure with a solution using fluorescein isothiocyanate (FITC)-conjugated PBP-9R by flow cytometry (FIG. 7A) and confocal microscopy (FIG. 7B).

FIGS. 8A and 8B show the results of comparing the gene-silencing effect of the SA-OP complex solution and the microstructure loaded with it on FABP4 and FABP5 by reverse transcription-quantitative polymerase chain reaction (RT-qPCR) (FIG. 8A) and western blot (FIG. 8B).

FIG. 9 shows the results of selecting leptin and adiponectin, which are factors related to insulin resistance, in order to verify the effect of FABP4 and FABP5 factor inhibition, and comparing the mRNA expression levels of leptin and adiponectin in the SA-OP complex solution and the microstructure loaded with it by RT-qPCR.

FIG. 10 shows the results of comparing the fluorescence intensity within the tissue upon subcutaneous injection and microstructure application at different time points using a diet-induced obesity model of C57BL/6. (vWAT: white fat (visceral fat), SWAT: white fat (subcutaneous fat)).

FIG. 11A shows the results of measuring body weight weekly after feeding 60% high-fat diet (HFD) to the C57BL/6 mice for 14 weeks and administering the complex (0.5 mg/kg of sh(FABP4/5)) three times a week for six weeks. FIG. 11B shows the results of measuring insulin resistance at week 7 and glucose resistance at week 8 after the six-week treatment.

FIG. 12A shows the results of confirming the protein expression levels and mRNA levels of FABP4 and FABP5 in the visceral fat after dissection after the six-week treatment and measuring the insulin and glucose resistance. FIG. 12B shows the results of confirming leptin and adiponectin mRNA levels in the visceral fat. FIG. 12C shows the results of confirming inflammatory factors in serum.

FIG. 13 shows the results of measuring triglyceride (TG) and free fatty acid (FFA) related to lipid metabolism in serum after the six-week treatment.

FIG. 14 shows the results of a histopathological analysis of the mouse liver through H&E staining after the six-week treatment.

FIG. 15 shows diagrams confirming the physicochemical properties of long-term-stored SA-OP (LMN). FIG. 15A shows schematic diagrams illustrating how the SA-OP is preserved in the LMN for long-term storage. The SA-OP in a solution may interact with each other and form aggregates after several days due to the innate charges generated by the gene and peptide, but it was confirmed that the HA backbone of the LMN prevents the SA-OP from interacting with each other and preserving the nanoparticle form.

FIG. 15B shows the results of optical measurement of the aggregates four weeks after the SA-OP preparation. FIG. 15C shows diagrams illustrating the size distribution of the SA-OP evaluated by DLS. FIG. 15D shows confocal laser scanning microscope images of the SA-OP (LMN) after four weeks of storage. FIG. 15E shows diagrams illustrating the fluorescence intensity of the gene and peptide inside the SA-OP (LMN) after four weeks of storage (left) and during four weeks of storage (right). FIG. 15F shows the results of the breaking force analysis of the SA-OP (LMN) at various storage time points.

FIG. 16 shows the confocal laser scanning microscope images of the SA-OP (LMN) during the 4-week storage period (sh(FABP4/5) (lower part of each period, red) and PBP-9R (upper part of each period, green)).

FIG. 17 diagrams illustrating the results of the in vitro cell uptake and treatment effect of the SA-OP stored for a long period of time. FIG. 17A shows histogram plots of sh(FABP4/5)-Cy5.5 of the SA-OP (SOL) and the SA-OP (LMN) at various time points. FIG. 17B shows the results of a relative average fluorescence intensity analysis of sh(FABP4/5)-Cy5.5 at different time points. The data are expressed as the mean±standard deviation (SD), n=3. FIG. 17C shows the results of the relative average fluorescence intensity analysis of PBP-9R-FITC at each time point. The data are expressed as the mean±SD, n=4. FIG. 17D shows a diagram illustrating the dual gene-silencing effect of the SA-OP stored for a long time in a solution and in the LMN. FIG. 17E shows the results of analyzing the leptin and adiponectin mRNA levels after treatment in the 3T3-L1 cells. The data are expressed as mean±SD, n=3.

FIG. 18 shows histogram plots for PBP-9R-FITC of the SA-OP (SOL) and the SA-OP (LMN) at various time points.

### MODES OF THE INVENTION

Hereinafter, the present invention will be described in more detail through examples. However, these examples are intended to describe the present invention in an illustrative manner, and the scope of the present invention is not limited to these examples.

### EXAMPLES AND EXPERIMENTAL EXAMPLES

### Manufacturing Example 1: Manufacturing of Gene sh(FABP4/5)

As a plasmid that induces the expression of two types of shRNA, a dual RNA PolIII cassette vector, psiRNA-DUO (InvivoGen, USA), was used, and the specific preparation process is described below.

A tube containing a frozen plasmid was spun to precipitate the DNA, and the obtained DNA was resuspended in 20 μl of sterile water to obtain a 1 μg/μl plasmid solution. The resuspended plasmid was stored at −20° C. The resulting plasmid was transformed into resuspended E. coli to perform a plasmid amplification process.

The psiRNA-DUO plasmid was treated with restriction enzyme BbsI (NEB, 2 units enzyme/μg plasmid DNA), and the large fragment (3180 bp) was eluted using 0.7% low-melting-point agarose gel. Then, the purified DNA fragment was diluted to obtain a 0.1 μg/μl solution (Gus cassette). In addition, the psiRNA-DUO plasmid was treated with Acc65I and HindIII together with the New England Biolabs (NEB) enzyme, NEBuffer 2, and bovine serum albumin (BSA), and the large fragment (3150 bp) was eluted using 0.7% low-melting-point agarose gel. The purified DNA fragment was diluted to obtain a 0.1 μg/μl solution (LacZ cassette).

To clone sh(FABP4/5) into psiRNA-DUO, a process of simultaneously treating psiRNA-DUO with Acc65I, HindIII, and BbsI and ligating the two resulting psiRNA-DUO fragments (HindIII/BbsI and BbsI/Acc65I) with two shRNA inserts was used, or a process of cloning the first shRNA insert and then cloning of the remaining second shRNA insert was used (Catalog #ksirna4-gz3).

The specific base sequences is shown below.

(SEQ ID NO: 1)
cctgcaggcg ttacataact tacggtaaat ggcccgcctg gctgaccgcc caacgacccc
cgcccattga cgtcaataat gacgtatgtt cccatagtaa cgccaatagg gactttccat
tgacgtcaat gggtggagta tttacggtaa actgcccact tggcagtaca tcaagtgtat
catatgccaa gtacgccccc tattgacgtc aatgacggta aatggcccgc ctggcattat
gcccagtaca tgaccttatg ggactttcct acttggcagt acatctacgt attagtcatc
gctattacca tgatgatgcg gttttggcag tacatcaatg ggcgtggata gcggtttgac
tcacggggat ttccaagtct ccaccccatt gacgtcaatg ggagtttgtt ttgactagta
aatcaacggg actttccaaa atgtcgtaac aactccgccc cattgacgca aatgggcggt
aggcgtgtac ggtgggaggt ctatataagc agagctcgtt tagtgaaccg tcagatcagc
ttcgaggggc tcgcatctct ccttcacgcg cccgccgccc tacctgaggc cgccatccac
gccggttgag tcgcgttctg ccgcctcccg cctgtggtgc ctcctgaact gcgtccgccg
tctaggtaag tttaaagctc aggtcgagac cgggcctttg tccggcgctc ccttggagcc
tacctagact cagccggctc tccacgcttt gcctgaccct gcttgctcaa ctctacgtct
ttgtttcgtt ttctgttctg cgccgttaca gatccaagcc accatggttt ctaagggaga
agaactcttt actggtgttg tcccaattct ggttgagctg gatggtgatg tgaatggcca
caaattctct gtgtctggtg aaggtgaagg agatgcaact tatggaaagc tgactctgaa
gttcatttgt acaacaggaa agctgccagt gccttggcca actctggtga ccaccctgac
ttatggtgtt caatgtttca gcagataccc tgaccacatg aagcagcatg acttctttaa
atctgcaatg ccagaaggtt atgttcagga gaggacaatc ttctttaagg atgatggaaa
ttataagaca agggcagaag tgaagtttga aggtgataca ctggttaaca gaattgagct
gaaaggcatt gattttaagg aagatggaaa cattctgggt cacaagctgg agtacaacta
taattctcac aatgtttaca ttatggcaga taagcagagg aatggaatta aggctaattt
caagattaga cacaacattg aggatggatc tgtccaactg gcagaccatt accagcagaa
cacccctatt ggtgatggcc cagttctcct cccagataat cactatctca gcactcaatc
tgctctgtcc aaagacccta atgagaaaag agaccacatg gtcctcctgg agtttgtgac
agcagcagga attactctgg gaatggatga gctgtacaag ggtaagtcac tgactgtcta
tgcctgggaa agggtgggca ggagatgggg cagtgcagga aaagtggcac tatgaaccca
ctagtttgac aattaatcat aagcatagta taatacaact cactatagca attgtactaa
ccttcttctc tttcctctcc tgacaggagg agccatcatg gccaaactca cttctgcagt
cccagtcctc acagcaaggg atgttgcagg ggctgtagag ttctggactg acagattagg
attctccaga gactttgttg aagatgattt tgctggtgtt gtcagagatg atgtcaccct
cttcatctca gcagttcagg accaagttgt ccctgacaac acccttgctt gggtctgggt
cagaggccta gatgagcttt atgcagaatg gtcagaagta gtcagcacaa atttcaggga
tgcctctggc ccagccatga cagaaattgg tgaacaacct tggggaaggg aatttgccct
cagagaccct gctggaaatt gtgtccattt tgtagctgag gaacaggact aaagctagaa
gctcgctttc ttgctgtcca atttctatta aaggttcctt tgttccctaa gtccaactac
taaactgggg gatattatga agggccttga gcatctggat tctgcctaat aaaaaacatt
tattttcatt gcaatgatgt atttaaatta tttctgaata ttttactaaa aagggaatgt
gggaggtcag tgcatttaaa acataaagaa atgaagagct agttcaaacc ttgggaaaat
acactatatc ttaaactcca tgaaagaagg tgaggctgca aacagctaat gcacattggc
aacagcccct gatgcctatg ccttattcat ccctcagaaa aggattcaag tagaggcttg
atttggaggt taaagttttg ctatgctgta ttttaattaa cgttctgcag tatttagcat
gccccaccca tctgcaaggc attctggata gtgtcaaaac agctggaaat caagtctgtt
tatctcaaac tttagcattt tgggaataaa tgatatttgc tatgctggtt aaattagatt
ttagttaaat ttcctgctga agctctagta tgataagtaa cttgacctaa gtgtaaagtt
gagatttcct tcaggtttat atagtcccta tcagtgatag agacctcggt cttcacctga
ggtttttcaa aagtagttga caattaatca tcggcatagt atatcggcat agtataatac
gactcactat aggagggcca ccatggtccg tcctgtagaa accccaaccc gtgaaatcaa
aaaactcgac ggcctgtggg cattcagtct ggatcgcgaa aactgtggaa ttgatcagcg
ttggtgggaa agcgcgttac aagaaagccg ggcaattgct gtgccaggca gttttaacga
tcagttcgcc gatgcagata ttcgtaatta tgcgggcaac gtctggtatc agcgcgaagt
ctttataccg aaaggttggg caggccagcg tatcgtgctg cgtttcgatg cggtcactca
ttacggcaaa gtgtgggtca ataatcagga agtgatggag catcagggcg gctatacgcc
atttgaagcc gatgtcacgc cgtatgttat tgccgggaaa agtgtacgta tcaccgtttg
tgtgaacaac gaactgaact ggcagactat cccgccggga atggtgatta ccgacgaaaa
cggcaagaaa aagcagtctt acttccatga tttctttaac tatgccggaa tccatcgcag
cgtaatgctc tacaccacgc cgaacacctg ggtggacgat atcaccgtgg tgacgcatgt
cgcgcaagac tgtaaccacg cgtctgttga ctggcaggtg gtggccaatg gtgatgtcag
cgttgaactg cgtgatgcgg atcaacaggt ggttgcaact ggacaaggca ctagcgggac
tttgcaagtg gtgaatccgc acctctggca accgggtgaa ggttatctct atgaactgtg
cgtcacagcc aaaagccaga cagagtgtga tatctacccg cttcgcgtcg gcatccggtc
agtggcagtg aagggcgaac agttcctgat taaccacaaa ccgttctact ttactggctt
tggtcgtcat gaagatgcgg acttacgtgg caaaggattc gataacgtgc tgatggtgca
cgaccacgca ttaatggact ggattggggc caactcctac cgtacctcgc attaccctta
cgctgaagag atgctcgact gggcagatga acatggcatc gtggtgattg atgaaactgc
tgctgtcggc tttaacctct ctttaggcat tggtttcgaa gcgggcaaca agccgaaaga
actgtacagc gaagaggcag tcaacgggga aactcagcaa gcgcacttac aggcgattaa
agagctgata gcgcgtgaca aaaaccaccc aagcgtggtg atgtggagta ttgccaacga
accggatacc cgtccgcaag gtgcacggga atatttcgcg ccactggcgg aagcaacgcg
taaactcgac ccgacgcgtc cgatcacctg cgtcaatgta atgttctgcg acgctcacac
cgataccatc agcgatctct ttgatgtgct gtgcctgaac cgttattacg gatggtatgt
ccaaagcggc gatttggaaa cggcagagaa ggtactggaa aaagaacttc tggcctggca
ggagaaactg catcagccga ttatcatcac cgaatacggc gtggatacgt tagccgggct
gcactcaatg tacaccgaca tgtggagtga agagtatcag tgtgcatggc tggatatgta
tcaccgcgtc tttgatcgcg tcagcgccgt cgtcggtgaa caggtatgga atttcgccga
ttttgcgacc tcgcaaggca tattgcgcgt tggcggtaac aagaaaggga tcttcactcg
cgaccgcaaa ccgaagtcgg cggcttttct gctgcaaaaa cgctggactg gcatgaactt
cggtgaaaaa ccgcagcagg gaggcaaaca ataatagcta gaggaagact ttttggaaaa
gattaaaaac ccgcttcggc gggttttttt atgcatgtga gcaaaaggcc agcaaaaggc
caggaaccgt aaaaaggccg cgttgctggc gtttttccat aggctccgcc cccctgacga
gcatcacaaa aatcgacgct caagtcagag gtggcgaaac ccgacaggac tataaagata
ccaggcgttt ccccctggaa gctccctcgt gcgctctcct gttccgaccc tgccgcttac
cggatacctg tccgcctttc tcccttcggg aagcgtggcg ctttctcata gctcacgctg
taggtatctc agttcggtgt aggtcgttcg ctccaagctg ggctgtgtgc acgaaccccc
cgttcagccc gaccgctgcg ccttatccgg taactatcgt cttgagtcca acccggtaag
acacgactta tcgccactgg cagcagccac tggtaacagg attagcagag cgaggtatgt
aggcggtgct acagagttct tgaagtggtg gcctaactac ggctacacta gaagaacagt
atttggtatc tgcgctctgc tgaagccagt taccttcgga aaaagagttg gtagctcttg
atccggcaaa caaaccaccg ctggtagcgg tggttttttt gtttgcaagc agcagattac
gcgcagaaaa aaaggatctc aagaagatcc tttgatcttt tctacggggt ctgacgctca
gtggaacgaa aactcacgtt aagggatttt ggtcatgttc ttaatcgata ctagtgctgc
agtatttagc atgccccacc catctgcaag gcattctgga tagtgtcaaa acagccggaa
atcaagtccg tttatctcaa actttagcat tttgggaata aatgatattt gctatgctgg
ttaaattaga ttttagttaa atttcctgct gaagctctag tacgataagt aacttgacct
aagtgtaaag ttgagatttc cttcaggttt atatagcttg tgcgccgcct gggtacctga
ggtttttcaa aagtagttga caattaatca tcggcatagt atatcggcat agtataatac
gactcactat aggagggcca ccatggaccc tgttgtgctg caaaggagag actgggagaa
ccctggagtg acccagctca acagactggc tgcccaccct ccctttgcct cttggaggaa
ctctgaggaa gccaggacag acaggcccag ccagcagctc aggtctctca atggagagtg
gaggtttgcc tggttccctg cccctgaagc tgtgcctgag tcttggctgg agtgtgacct
cccagaggct gacactgtgt aaccctaagc ttctagactt aattaa

### Manufacturing Example 2: Manufacturing of Peptide-Based Carrier (PBP-9R) Capable of Adipocyte-Targeting

The carrier consists of a prohibitin-binding protein (PBP) peptide sequence (adipocyte-targeting sequence) that is capable of selectively targeting adipocytes and a sequence including nine D-form arginine residues (RRRRRRRRR, 9R) that increases the ease of entry into cells with positive charges. The monomer of the peptide carrier consists of ‘C-PBP-RRRRRRRRR-C’ (Cys Lys Gly Gly Arg Ala Lys Asp Arg Arg Arg Arg Arg Arg Arg Arg Cys) (SEQ ID NO: 2). The molecular weight is 2341, and it was purchased from Peptron Inc. The lyophilized peptide was dissolved in deionized water and stored at −20° C.

### Manufacturing Example 3-1: Formation of Microstructure of Nanoparticles Using Micromolding Technique

20% (w/v) of HA was mixed with the adipocyte-targeting peptide-based gene/carrier complex to form nanoparticles. Using micromolding, the mixture was loaded into a polydimethylsiloxane (PDMS) mold manufactured using a master template. Thereafter, a microstructure loaded with nanoparticles was generated using a centrifuge or a vacuum device. After drying at room temperature, the formation of a soluble interlocking microstructure was confirmed.

### Manufacturing Example 3-2: Fabrication of Soluble Microstructure Array Mold and Array

An interlocking microstructure was fabricated in the form of a 14×14 array. The structure of the interlocking microstructure had a height of 700 μm, mid-diameter of 400 μm, and base diameter of 250 μm. A non-dissolvable HTL (high-temperature liquid) resin employed as the backbone matrix of the 3D printed mold. The 3D printed mold was then casted using PDMS (SYLGARD™ 184 Silicone Elastomer, DOW Corning) at a 10:1 ratio to fabricate a negative mold, which was then annealed at 80° C. for one hour. Next, the microstructure PDMS mold was plasma-treated for 10 seconds (mid). The plasma treatment created a hydrophilic surface, allowing an HA (20%) solution to enter into the PDMS microstructure mold cavity.

### Experimental Example 1. Experimental Method

### (1) Mechanical Test of Microstructure

A mechanical strength test for dissolving microstructures was performed using a Zwick Roell Z0.5 Materials Testing Machine (Zwick Roell). HA was used to fabricate a soluble microstructure. A microneedle was installed on an aluminum plate with the tip of the microneedle facing upward. The tip of the microstructure was compressed at a constant speed of 10 mm/sec. The distance and the force were recorded on the materials testing machine until the preset force of 3 N was reached.

### (2) Skin Insertion Test of Microneedle

To determine whether the microneedle array could penetrate the skin, the microneedle array was compressed for five minutes on the mouse skin collected from obese mice. To confirm the penetration of histological specimens, mouse skin samples were collected from the obese mice. After compressing the microneedle on each skin sample for five minutes, the skin samples were fixed with 4% paraformaldehyde and embedded in paraffin blocks before sectioning. The paraffin sections were stained by H&E staining.

### (3) 3T3-L1 Adipocyte Differentiation

3T3-L1 cells were purchased from the American Type Culture Collection (ATCC). High-glucose Dulbecco's Modified Eagle's Medium (DMEM) was purchased from WelGENE. DMEM containing 1% penicillin-streptomycin (100 U/ml) and 10% fetal bovine serum (FBS, WelGENE) was used for cell culture. 3T3-L1 cells were cultured at 37° C. and 5% CO₂. 3T3-L1 preadipocyte cells were cultured three times a week and induced to differentiation was induced for 72 hours using 1 μM dexamethasone, 0.5 mM 3-isobutyl-1-methylxanthine (IBMX), 10 μg/ml insulin (multiple daily injection (MDI) solution) for 72 hours. The medium was removed and replaced with complete medium containing 10 μg/ml insulin. The medium was exchanged with fresh medium every two days.

### (4) CCK-8 Assay

Mature adipocytes were cultured in 6-well plates for 48 h. Thereafter, cells were treated with various amounts of SA-OP with or without an HA solution, and then a Cell Counting Kit-8 (CCK-8) solution was added for 30 minutes. Relative cell viability compared to the control group was measured at 450 nm using a UV/vis spectrophotometer (Infinite 200 PRO microplate Reader, TECAN).

### (5) Flow Cytometry Analysis

sh(FABP4/5)/FITC-PBP9R (Peptron Inc., Korea) was prepared at room temperature for 30 minutes. Cellular uptake was measured to compare sh(FABP4/5)/FITC-PBP9R with sh(FABP4/5)/FITC-PBP9R-loaded microneedles. FITC-PBP-9R was used to measure the mean fluorescence intensity of cellular uptake by flow cytometry. After 1, 4, 24, and 48 hours of transfection, mature adipocytes were taken following trypsinization, and a single-cell suspension was prepared in FBS containing a fluorescence-activated single cell sorting (FACS) buffer (2% FBS and 0.02% sodium azide in phosphate-buffered saline (PBS)). Cellular uptake of sh(FABP4/5)/FITC-PBP-9R was evaluated using FACSCalibur (BD Biosciences), and the data were analyzed using CellQuest Pro. Adipocytes were gated by forward and side scatter, and 10,000 events were measured per sample. Internalization of sh(FABP4/5)/FITC-PBP-9R was performed using a confocal microscope. Adipocytes were seeded on glass coverslips in 6-well plates and then differentiated and matured. After 1, 6, 12, and 24 hours of transfection, cells were washed with PBS and deionized-double distilled water (3DW) and mounted using DAPI Fluoromount-G (Southern Biotech) to stain nuclei. Each image was visualized using a Tata Consultancy Services (TCS) Service Pack 5 (SP5) Carl Zeiss confocal laser scanning microscope (Leica, Hanyang University).

(6) RNA Isolation and qPCR (In Vitro)

After transfection of SA-OP complex solution and microneedle for 48 hours, total RNA was isolated from mature adipocytes of each group using RNeasy mini kit (Qiagen). cDNA samples for RT-qPCR were obtained using iScript cDNA Synthesis Kit (Bio-Rad), and the samples were measured with SYBR Green (Bioline) using Thermo Fisher 7500 Fast Real-Time PCR System (Thermo Fischer). The relative mRNA levels of FABP4, FABP5, adiponectin, and leptin to glyceraldehyde 3-phosphate dehydrogenase (GAPDH) were calculated using the delta-delta (ΔΔ) Ct method. Each primer was purchased from Bioneer. The specific primer sequence list is shown in Table 1.

TABLE 1

| | Forward | Reverse |
|---|---|---|
| Mouse | CATCACTGCCACCCAGAAG | ATGCCAGTGAGCTTCCCGTTC |
| GADPH | ACTG (SEQ ID NO: 11) | AG (SEQ ID NO: 12) |
| Mouse FABP4 | TGAAATCACCGCAGACGAC | GCTTGTCACCATCTCGTTTTCT |
| | AGG (SEQ ID NO: 3) | C (SEQ ID NO: 4) |
| Mouse FABP5 | TGGTTTACCCAGGATCATTC | CCTGAAGAATACCAGAGAGCT |
| | C (SEQ ID NO: 5) | T (SEQ ID NO: 6) |
| Mouse Leptin | TGAGTTTGTCCAAGATGGA | GCCATCCAGGCTCTCTGG (SEQ |
| | CC (SEQ ID NO: 7) | ID NO: 8) |
| Mouse | CAATGTACCCATTCGCTTTA | CATACACCTGGAGCCAGACT |
| Adiponectin | CT (SEQ ID NO: 9) | (SEQ ID NO: 10) |

### (7) Protein Isolation and Western Blot (In Vitro)

Mature adipocytes were treated with the SA-OP complex solution and microneedles for 48 hours. The cells were lysed by using a radioimmunoprecipitation assay (RIPA) buffer containing protease inhibitor cocktail (Roche) and then vortexed. After incubation on ice for 15 minutes, the cell suspension was centrifuged at 14,800 rpm for 15 minutes at 4° C. The protein concentration was measured by bicinchoninic acid (BCA) assay using the supernatant. The samples were loaded onto polyacrylamide gels, and sodium dodecyl sulfate-polyacrylamide gel electrophoresis (SDS-PAGE) was performed, and then the gels were transferred to polyvinylidene difluoride (PVDF) membranes (Bio-Rad). The gels were treated with antibodies including FABP4, FABP5, and housekeeping genes (β-actin) overnight on the PVDF membrane. Antibody binding to the samples on the PVDF membranes was visualized by electrochemiluminescence (ECL, Millipore) and detected by a ChemiDoc™ XRS+system (Bio-Rad).

### (8) Protein Isolation and Enzyme-Linked Immunosorbent Assay (ELISA) (In Vitro)

Mature adipocytes were treated with the SA-OP complex solution and microneedles for 48 hours. The protein concentrations of TNF-α, IL-6, IL-1β, and MCP-1 in the cell medium were measured by ELISA (Invitrogen). Mature adipocytes were lysed with a 1× lysis buffer and vortexed, and then the protein concentration was measured by BCA assay to control the cell number.

### (9) Diet-Induced Obesity Mouse Model

To create an obesity and metabolic syndrome model, six-week-old male C57BL/6J mice were obtained from Orient Bio. The use of experimental mice was approved by the Institutional Animal Care and Use Committee of Hanyang University (2020-0961). Mice were randomly assigned to one of four groups (n=5 per group). C57BL/6 mice were fed a normal diet for the first week (Central Lab Animal, Inc.). Then, the diet was mixed with 10% high-fat diet (HFD) (60% of calories from fat). The proportion of HFD in the total diet was gradually increased for six weeks, and then the mice were fed only HFD for eight weeks. After 20 weeks, the mice became obese and developed insulin resistance, with an approximate body weight of 50 g and a glucose level of >200 mg/dL.

### (10) Insulin Tolerance Test (ITT) and Glucose Tolerance Test (GTT)

In order validate the obesity and insulin resistance models, the initial blood glucose levels were measured at 6-hour post-fasting, using the Accu-Chek Active model GC kit (RocheDiagonostics GmbH). Insulin (0.75 units/kg) was injected intraperitoneally. Blood samples were collected from the tail vein at 0, 30-, 60-, 90- and 120-min post-injection for the ITT. After six weeks of treatment with the SA-OP complex via subcutaneous administration (S.C.) and microneedles (MN), the obesity and metabolic syndrome models were applied. The initial blood glucose levels were measured at 6-hour post-fasting using a blood glucose meter. Insulin (0.75 units/kg) or glucose (2 g/kg) was injected intraperitoneally. Blood samples were collected from the tail vein at 0, 30-, 60-, 90- and 120-min post-injection for the ITT. Blood samples were collected from the tail vein at 0, 15, 30-, 60-, 90- and 120-min post-injection for the GTT.

### (11) Biodistribution

After the obesity and insulin resistance modeling, sh(FABP4/5)/FITC-PBP-9R was injected into mice via S.C. and MN. FITC fluorescence intensity was measured in the liver, kidneys, spleen, heart, lungs, visceral white adipose tissue (vWAT), and subcutaneous white adipose tissue (sWAT) using FOBI (CELLGENTEK, South Korea), and dissected at 1, 6, 24, 48, and 72 hours. The intensity was analyzed by software and visualized as rainbow colors.

(12) RNA Isolation and qPCR (In Vitro)

After six-week treatment, mice were sacrificed, perfused with PBS, and tissues were harvested. Thereafter, the tissues were homogenized, and total RNA was isolated from tissues of each group using an RNeasy mini kit, and cDNA was obtained by synthesizing via an iScript cDNA Synthesis Kit. Samples were measured using the Thermo Fisher 7500 Fast Real-Time PCR System. The relative mRNA levels of FABP4, FABP5, adiponectin, and leptin to GAPDH were calculated using the ΔΔCt method.

### (13) Protein Isolation and Western Blot (In Vitro)

sh(FABP4/5)/PBP-9R was administered to the abdominal fat pad three times a week for six weeks. After perfusion of 5 ml PBS through the left ventricle, the tissues were harvested and incubated on ice before homogenization. Thereafter, the tissues were then homogenized with Reporter lysis 1× buffer containing 0.1 mM phenylmethylsulfonyl fluoride (PMSF) protease inhibitor. The homogenized tissue samples were centrifuged at 14,800 rpm for 15 minutes at 4° C. After collecting the supernatant, the samples were loaded onto a polyacrylamide gel and subjected to SDS-PAGE, and the gels were transferred to a PVDF membrane (Bio-Rad). The gels were treated with antibodies including FABP4, FABP5, and housekeeping genes (β-actin) overnight on the PVDF membrane. Antibody binding to the samples on the PVDF membranes was visualized by ECL (Millipore) and detected by a ChemiDoc™ XRS+system (Bio-Rad).

### (14) Ex Vivo Analysis of Tissues by Immunohistochemistry (IHC)

Adipose tissues were harvested and fixed with 4% paraformaldehyde in 1×PBS, and then embedded in paraffin blocks before sectioning. After sectioning in 8 μm, the blocks were deparaffinized and gradually rehydrated in ethanol. Anti-FABP4 and anti-FABP5 antibodies were incubated overnight in epididymal adipose tissues. Alexa488-conjugated anti-rabbit antibodies were incubated for two hours. Coverslips were mounted on stained slides using Dako Fluorescence Mounting Medium (DAKO, Denmark) and scanned with AxioScan.Z1 (Zeiss, Germany).

### (15) Protein Separation and ELISA (In Vivo)

After diet-induced obesity (DIO) modeling, sh(FABP4/5)/PBP-9R was injected into the abdominal fat pad via S.C. and MN three times a week for six weeks. Whole blood samples were collected from the right ventricle of the treated mice. The extracted blood samples were incubated at room temperature for 30 minutes, the covers were opened, and the samples were coagulated. Thereafter, the samples were centrifuged at 1500 g for 20 minutes at 4° C. The samples were stored in a deep freezer with 0.1 mM PMSF protease inhibitor. The expression levels of inflammatory cytokine proteins such as TNF-α, IL-6, IL-1β, and MCP-1 were measured using their respective ELISA kits.

### (16) Liver Function Test (LFT) and Liver Histopathology

After centrifuging blood samples at 3000 rpm and 4° C. for 20 minutes, the contents of triglyceride (TG) and free fatty acid (FFA) were measured using the Triglyceride Assay Kit and Free Fatty Acid Assay Kit (abcam) through serum. The effects of lipid metabolism were confirmed. Thereafter, serum samples were used to measure aspartate transaminase (AST) and alanine transaminase (ALT) to confirm drug-related liver toxicity. Liver tissue samples were collected from the treated mice, fixed with 4% paraformaldehyde, and embedded in paraffin blocks. Each block was distinguished and stained by H&E staining.

### (17) Statistical Analysis

At least three replicates were used in all in vitro studies. To achieve statistical significance in in vivo studies, n=5 per group was used. GraphPad Prism version 8.0 for Windows (GraphPad Software) was used for statistical analysis. Statistical significance of comparisons between two groups was calculated using a two-sided Student's t-test, and a p-value of less than 0.05 was considered significant. Results from all other experiments were analyzed using two-way analysis of variance through Bonferroni's correction and two-sided test for multiple comparisons between subgroups.

### Experimental Example 2: Agarose Gel Electrophoresis and Complex Size Measurement of Stability in Acidic Solution at Different Time Points

A complex was formed with 1 μg of sh(FABP4/5) with PBP-9R at a weight ratio of 1:3 (30 minutes at room temperature). Incubation was performed in an HA (20%) solution for the corresponding time to measure the stability at different time points (1, 6, 24, and 72 hours). The stability of the complex at different pH levels and time points were confirmed by electrophoresis in 0.8% (w/v) agarose gel in 0.5× tris-borate-ethylenediaminetetraacetic acid (TBE) buffer at 100 V for 20 minutes. The sh(FABP4/5) gene was incubated with PBP-9R at room temperature for 30 minutes to form a complex. Incubation was performed in an HA (20%) solution for the corresponding time to measure the stability at different time points (1, 6, 24, and 72 hours). Thereafter, the total volume was adjusted to 800 μl with deionized water, and the size of the complex was measured using a Zeta sizer-ZS (Malvern) machine.

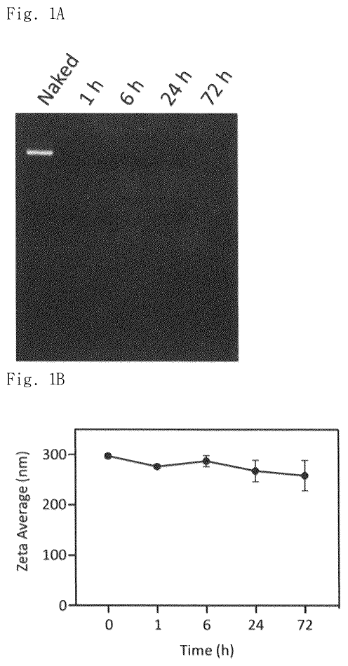

As a result, it was confirmed that the complex was not sensitive to low pH and was stable for up to 72 hours at room temperature (see FIGS. 1A and 1B). These results mean that the complex may be mixed with a biocompatible polymer to fabricate microneedles.

### Experimental Example 3: Agarose Gel Electrophoresis for Measuring Stability in Serum

A complex was formed with 1 μg of sh(FABP4/5) and PBP-9R at a weight ratio of 1:3 (at room temperature for 30 minutes). The sh(FABP4/5) gene and the complex were each incubated in mouse serum at 37° C. for 30 minutes. The serum stability of the gene alone and that of the complex was compared by electrophoresis in a 0.8% (w/v) agarose gel in 0.5×TBE buffer at 100 V for 20 minutes.

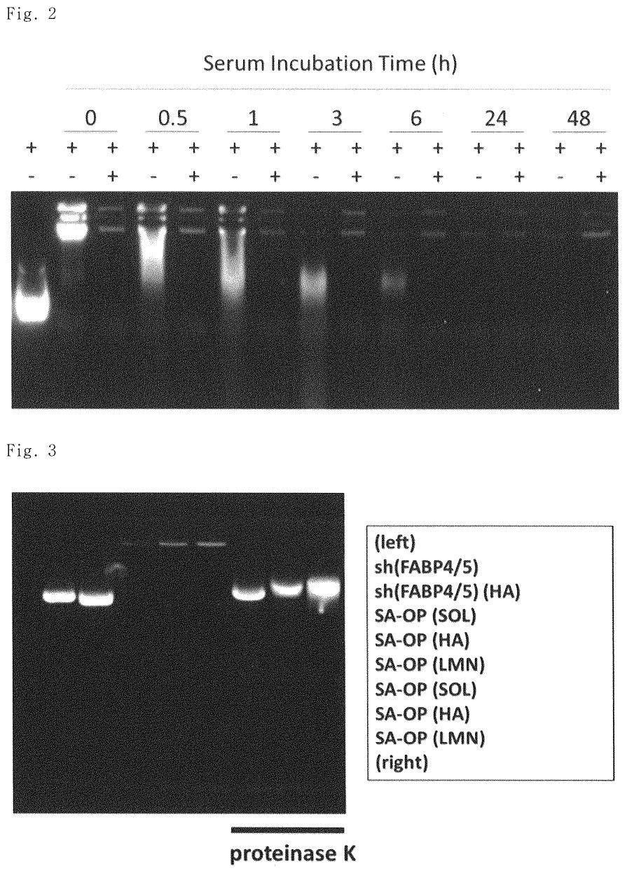

As a result, it was confirmed that the complex was stable in serum for 48 hours (see FIG. 2). These results mean that the complex can be applied as a subcutaneous injection or transdermal formulation.

### Experimental Example 4: Agarose Gel Electrophoresis to Confirm Stability after Microstructure Fabrication

A complex was formed with sh(FABP4/5) and PBP-9R at a weight ratio of 1:3 (30 minutes at room temperature). Thereafter, a soluble interlocking microstructure was formed using centrifugation using a micromolding technique. Then, the volume was adjusted to load sh(FABP4/5) onto the agarose gel. Proteinase K was used to confirm whether the gene in the complex was safely present. The amount was confirmed through the band position and thickness by electrophoresis in a 0.8% (w/v) agarose gel in a 0.5×TBE buffer solution at 100 V for 20 minutes.

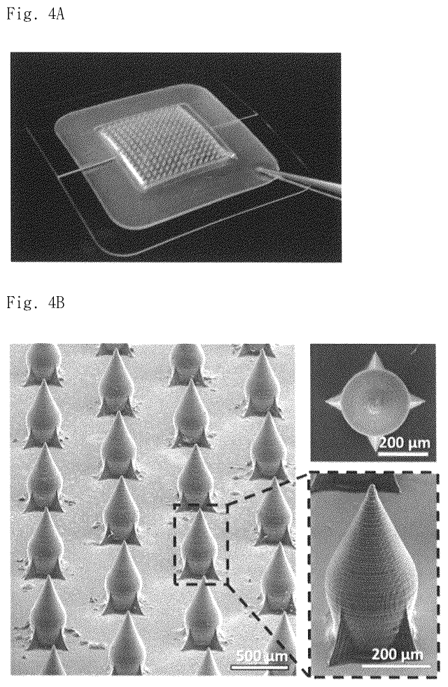

As a result, it was confirmed that the complex was stable even after fabricating the microstructure (see FIG. 3). These results mean that the microstructure can be used as a therapeutic agent.

### Experimental Example 5: Confirmation of Microstructure Properties

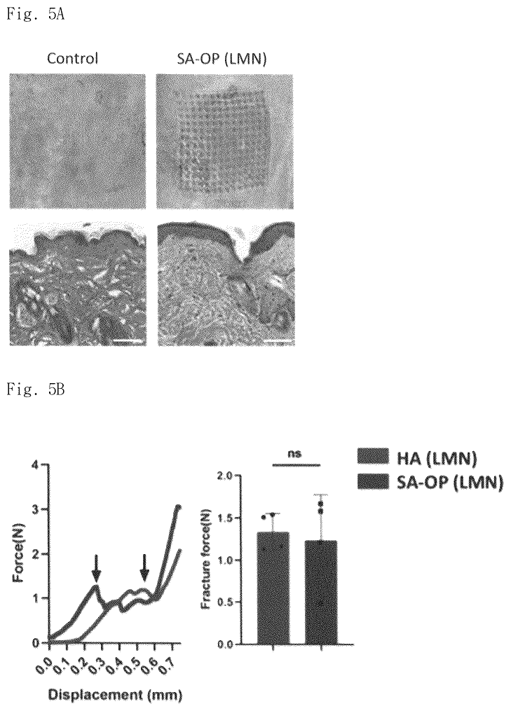

A complex was formed with sh(FABP4/5) and PBP-9R at a weight ratio of 1:3 (30 minutes at room temperature). Thereafter, a soluble interlocking microstructure was formed using centrifugation with a micromolding technique, and its properties were confirmed using an optical microscope and a scanning electron microscope (see FIG. 4).

As a result, it was confirmed that the shape of the needle was well formed according to the mold shape after the microstructure was fabricated. These results mean that there was no problem with the ratio and fabrication method.

### Experimental Example 6: Physical Properties of Microstructure and Skin Penetration Experiment

A complex was formed with sh(FABP4/5) and PBP-9R at a weight ratio of 1:3 (30 minutes at room temperature). Thereafter, a soluble interlocking microstructure was formed using centrifugation using a micromolding technique. The resulting microstructure was applied to the extracted obese mouse skin, fixed in 4% paraformaldehyde, and photographed under a microscope through H&E staining.

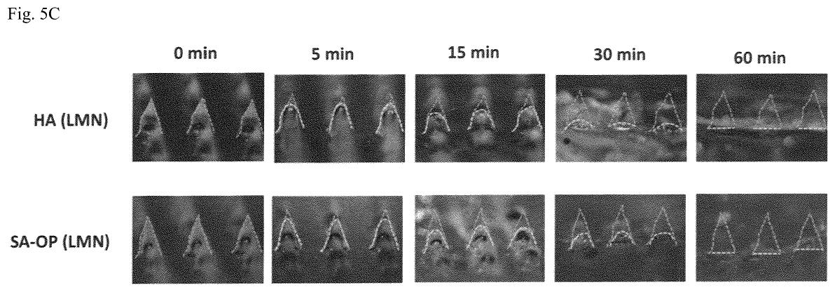

As a result, the actual penetration depth was approximately 400 μm, confirming that the microstructure penetrated a part of the dermal layer (see FIG. 5A). These results mean that the microstructure can penetrate the dermis of the mouse skin and deliver the drug.

To numerically determine the physical properties, the compressive strength was measured using a Zwick Roell Z0.5 Materials Testing Machine (ZwickRoell). The tip of the microstructure was compressed at a speed of 10 mm/see, and the change of the force and travel distance were shown as a graph.

As a result, it was confirmed that the strength of the microstructure was approximately 1.2 N, regardless of the presence or absence of HA (see FIG. 5B). These results mean that the microstructure has sufficient strength to penetrate the skin.

The microstructure penetrating the skin should enable the continuous release and absorption of SA-OP into the body. The time it takes for the microstructure to be dissolved under controlled conditions at 37° C. was measured. As a result, it was confirmed that the microstructure was dissolved after one hour (see FIG. 5C).

### Experimental Example 7: Measurement of Transdermal Water Loss and In Vitro Skin Absorption Experiment

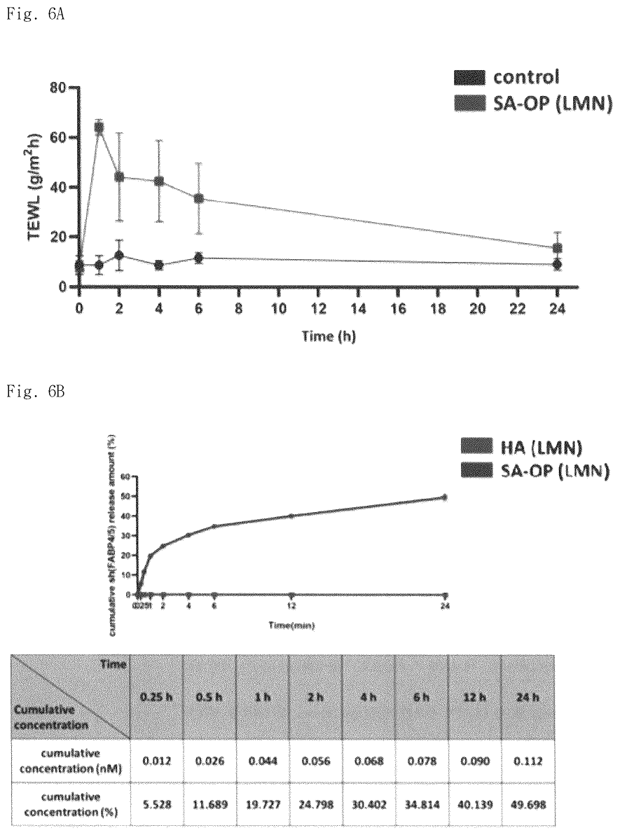

A complex was formed with sh(FABP4/5) and PBP-9R at a weight ratio of 1:3 (30 minutes at room temperature). Thereafter, a soluble interlocking microstructure was formed using centrifugation using a micromolding technique. The resulting microstructure was applied to mouse skin, and the time it took for the skin to recover was measured. It was confirmed that the skin recovered after 24 hours (see FIG. 6A). In addition, the soluble interlocking microstructure was applied to pig skin, and the amount of gene release was measured by sampling at different time points using a Franz diffusion cell device.

As a result, it was confirmed that 50% of the drug was released through the skin for 24 hours (see FIG. 6B). These results confirmed the actual drug permeation rate of the microneedle, so that the application time of the drug may be determined.

### Experimental Example 8: Comparative Analysis of PBP-9R Binding Ability to Differentiated 3T3-L1 Adipocytes (Differentiated Adipocytes) in Solution and Microstructure

FITC fluorescence-conjugated PBP-9R and sh(FABP4/5) were allowed to react at room temperature for 30 minutes, and then a microstructure was formed using the micromolding technique. The same amount of complex each in the microstructure and the solution was applied to differentiated adipocytes, and an analysis was performed by FACS. In addition, the penetration into the nucleus was compared using confocal microscopy.

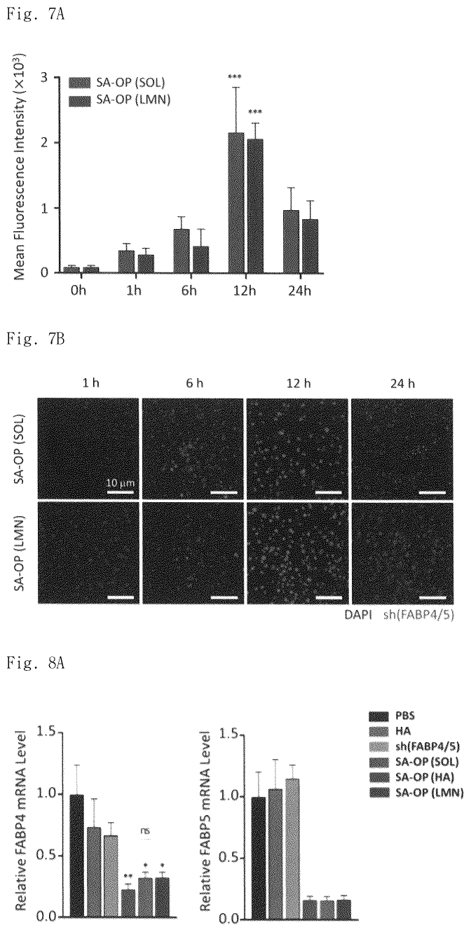

As a result, it was confirmed that the adipocyte binding ability patterns at different time points were similar in the solution and in the microstructure (see FIGS. 7A and 7B). These results mean that the HA and the microneedle fabrication process did not affect the adipocyte binding ability of the complex.

### Experimental Example 9: FABP4, FABP5 mRNA, and Protein Measurement (Differentiated Adipocyte RNA Isolation and Real-Time PCR, Protein Isolation and Western Blot)

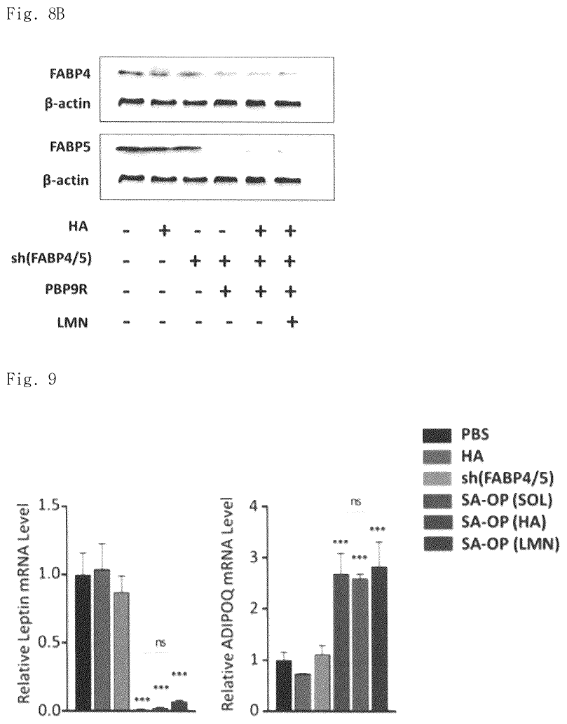

8×10⁴ mouse-derived preadipocytes (3T3-L1) per well of a cell culture plate were cultured in a six-well plate using DMEM, and differentiated by treating with 1 μM dexamethasone, 0.5 mM IBMX, and 10 μg/ml insulin for 72 hours. Thereafter, the fat droplet size was continuously increased with a culture medium containing 10 μg/ml insulin. Cells were treated with the sh(FABP4/5)/PBP-9R complex in a six-well plate for 48 hours. Thereafter, the cells were uniformly broken using the RNeasy mini kit (Qiagen), and only RNA was isolated. The isolated RNA was subjected to a reaction with reverse transcriptase using iScript cDNA synthesis kit (Bio-Rad) to synthesize complementary cDNA for each 1 μg of RNA from each group. Thereafter, the amounts of FABP4 and FABP5 mRNA relative to the endogenous control, GAPDH, were measured by real-time PCR using SYBR green (Bioline) (see FIG. 8A). The forward and reverse primers for FABP4 were 5′-TGAAATCACCGCAGACGACAGG-3′ (SEQ ID NO: 3) and 5′-GCTTGTCACCATCTCGTTTTCTC-3′ (SEQ ID NO: 4), respectively, and the forward and reverse primers for FABP5 were 5′-TGGTTTACCCAGGATCATTCC-3′ (SEQ ID NO: 5) and 5′-CCTGAAGAATACCAGAGAGCTT-3′ (SEQ ID NO: 6), respectively.

Differentiated adipocytes in a six-well plate were treated with sh(FABP4/5)/PBP-9R complex for 48 hours. Thereafter, they were treated with a RIPA buffer, and the protein-containing supernatant was obtained by centrifugation at 16,800 rpm and 4° C. for 15 minutes. After allowing the protein sample to react with the Laemmli buffer for 10 minutes, 10% SDS-PAGE electrophoresis was performed, and the expression levels of FABP4 and FABP5 proteins were measured using PVDF membranes (Millipore) and a trans-blot turbo transfer system (Bio-rad) with anti-FABP4 and FABP5 antibodies (see FIG. 8b).

As a result, it was confirmed that the gene inhibition efficacy of the solution and the microstructure was similar. These results mean that the presence or absence of HA and the microstructure fabrication process did not affect the gene inhibition efficacy of the complex.

### Experimental Example 10: Measurement of Leptin and Adiponectin mRNA (Isolation of Differentiated Adipocyte RNA and Real-Time PCR)

8×10⁴ mouse-derived preadipocytes (3T3-L1) per well of a cell culture plate were cultured in a six-well plate using DMEM, and differentiated by treating with 1 μM dexamethasone, 0.5 mM IBMX, and 10 μg/ml insulin for 72 hours. Thereafter, the fat droplet size was continuously increased with a culture medium containing 10 μg/ml insulin. Cells were treated with the sh(FABP4/5)/PBP-9R complex in a six-well plate for 48 hours. Thereafter, the cells were uniformly broken using the RNeasy mini kit (Qiagen), and only RNA was isolated. The isolated RNA was subjected to a reaction with reverse transcriptase using iScript cDNA synthesis kit (Bio-Rad) to synthesize complementary cDNA for each 1 μg of RNA from each group.

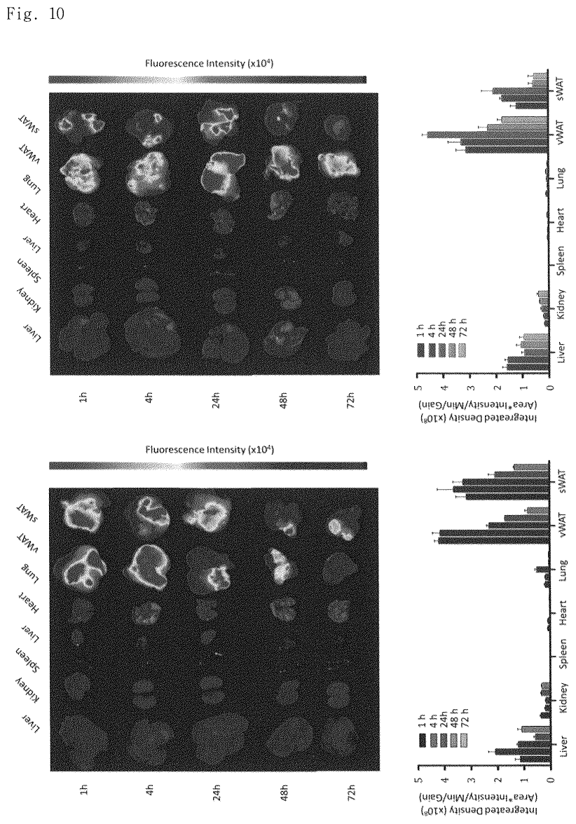

Thereafter, the amounts of leptin and adiponectin mRNA relative to the endogenous control, GAPDH, were measured by real-time PCR using SYBR green (Bioline) (see FIG. 9). The forward and reverse primers for leptin were 5′-TGAGTTTGTCCAAGATGGACC-3′ (SEQ ID NO: 7) and 5′-GCCATCCAGGCTCTCTGG-3′ (SEQ ID NO: 8), respectively, and the forward and reverse primers for adiponectin were 5′-CAATGTACCCATTCGCTTTACT-3′ (SEQ ID NO: 9) and 5′-CATACACCTGGAGCCAGACT-3′ (SEQ ID NO: 10), respectively.

As a result, it was confirmed that the gene inhibition efficacy of the solution and the microstructure was similar. These results mean that the presence or absence of HA and the microstructure fabrication process did not affect the gene inhibition efficacy of the complex.

### Experimental Example 11: Ex Vivo Biodistribution

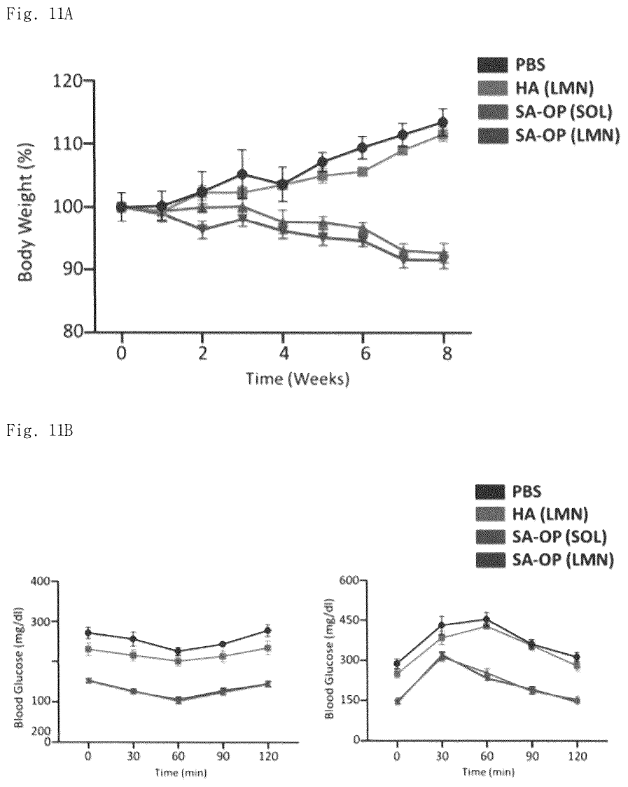

A DIO model was created by feeding six-week-old C57BL/6 mice a 60% HFD for 14 weeks. PBP-9R and sh(FABP4/5) conjugated with FITC fluorescence were allowed to react at room temperature for 30 minutes, and then a microstructure was formed using the micromolding technique. The solution was applied subcutaneously, and the microstructure was also applied to the abdomen, and the mice were dissected at different time points (1, 4, 24, 48, and 72 hours). Thereafter, images were taken with FOBI (CELLGENTEK) to analyze the fluorescence intensity (see FIG. 10).

The SA-OP (SOL) rapidly entered the systemic circulation one hour after inoculation in the vWAT abundantly expressing the prohibitin protein, as indicated by the increasing fluorescence intensity. The maximum fluorescence intensity was measured at 4 hours after inoculation in the vWAT, and the SA-OP signal decreased thereafter and ultimately disappeared after 72 hours. In contrast, the SA-OP released from the microstructure exhibited a distinct biodistribution pattern due to the continuous release by dissolution of the microstructure. The fluorescence intensity of the SA-OP-loaded microstructure gradually increased in the vWAT and reached a maximum at 24 hours. In addition, the accumulated SA-OP persisted longer in the vWAT and exhibited a significant value at 48 hours.

As a result, it was confirmed that the visceral fat targeting effects of the solution and the microstructure were similar. These results mean that the presence or absence of HA and the microstructure fabrication process did not affect the visceral fat targeting efficacy of the complex.

### Experimental Example 12: Confirmation of Body Weight Reduction and Improvement of Insulin Resistance and Glucose Resistance

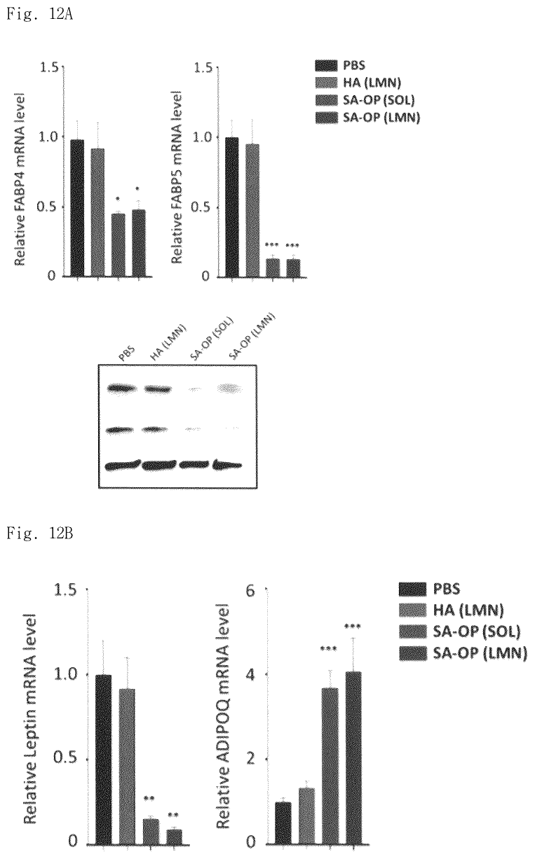

A DIO model was created by feeding six-week-old C57BL/6 mice a 60% HFD for 14 weeks. PBP-9R and sh(FABP4/5) were allowed to react at room temperature for 30 minutes, and then a microstructure was formed using the micromolding technique. The solution was applied subcutaneously, and the microstructure was also applied to the abdomen for six weeks. Body weight was measured daily and analyzed on a weekly basis (see FIG. 11A).

After applying insulin to the abdomen while fasting for six hours, blood glucose levels in the tail vein were measured at intervals of 0, 30, 60, 90, and 120 minutes using the Accu-Chek Active model GC kit (Roche Diagnostics GmbH). After one week, glucose was applied to the abdomen while fasting for six hours, and then blood glucose levels in the tail vein were measured using an Accu-Chek Active model GC kit at intervals of 0, 15, 30, 60, 90, and 120 minutes (see FIG. 11B).

As a result, it was confirmed that the body weight reducing effect and the insulin resistance and glucose resistance relieving effects were similar between the solution and the microstructure. These results mean that the presence or absence of HA and the microstructure fabrication process did not affect the efficacy of the complex.

### Experimental Example 13: Ex Vivo Sampling, Protein Isolation of Visceral Fat and Western Blot, RNA Isolation and Real-Time PCR, and Serum Isolation and ELISA

A DIO model was created by feeding six-week-old C57BL/6 mice a 60% HFD for 14 weeks. PBP-9R and sh(FABP4/5) were allowed to react at room temperature for 30 minutes, and then a microstructure was formed using the micromolding technique. The solution was applied subcutaneously, and the microstructure was also applied to the abdomen for six weeks. Visceral fat and serum were obtained by dissecting the laboratory animals. Organ tissue samples were physically pulverized, treated with Reporter lysis 5× buffer (Promega) and 0.1 mM PMSF, and centrifuged at 16,800 rpm and 4° C. for 30 minutes to obtain protein-containing supernatants. The protein samples were allowed to react with the Laemmli buffer for 10 minutes, and 10% SDS-PAGE electrophoresis was performed, and then the protein expression levels of FABP4 and FABP5 were measured using PVDF membranes (Millipore) and a trans-blot turbo transfer system (Bio-rad) with anti-FABP4 and FABP5 antibodies. In addition, some organ samples were homogeneously disrupted with RLT buffer (Qiagen), RNA was obtained, cDNA was synthesized using iScript cDNA synthesis kit, and the amounts of FABP4 and FABP5 mRNA relative to GAPDH, an endogenous control, were measured by real-time PCR.

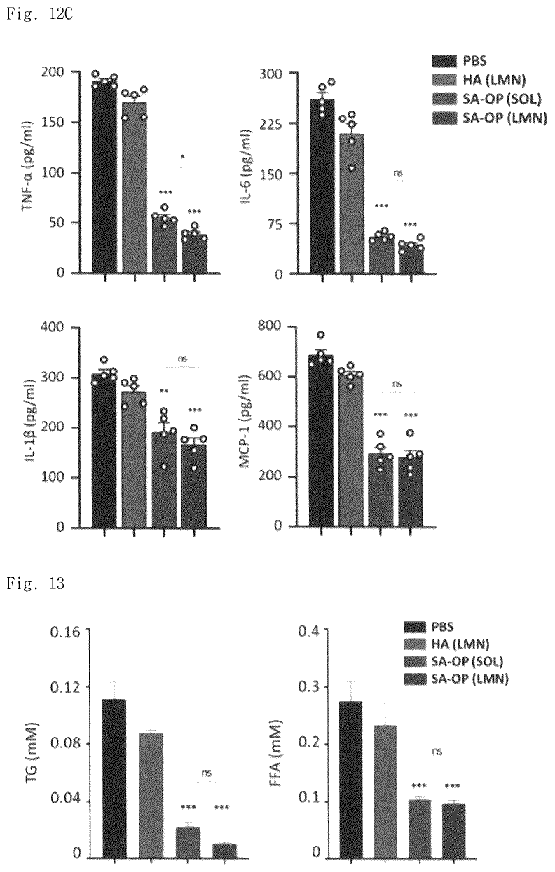

The forward and reverse primers for FABP4 were 5′-TGAAATCACCGCAGACGACAGG-3′ (SEQ ID NO: 3) and 5′-GCTTGTCACCATCTCGTTTTCTC-3′ (SEQ ID NO: 4), respectively, and the forward and reverse primers for FABP5 were 5′-TGGTTTACCCAGGATCATTCC-3′ (SEQ ID NO: 5) and 5′-CCTGAAGAATACCAGAGAGCTT-3′ (SEQ ID NO: 6), respectively. (see FIG. 12A).

As a result, it was confirmed that the gene inhibition efficacy in the visceral fat of the solution and the microstructure was similar. These results mean that the presence or absence of HA and the microstructure fabrication process did not affect the gene inhibition efficacy of the complex.

In addition, the amounts of leptin and adiponectin mRNA, which are factors related to insulin resistance, were measured through real-time PCR. The forward and reverse primers for leptin were 5′-TGAGTTTGTCCAAGATGGACC-3′ (SEQ ID NO: 7) and 5′-GCCATCCAGGCTCTCTGG-3′ (SEQ ID NO: 8), respectively, and the forward and reverse primers for adiponectin were 5′-CAATGTACCCATTCGCTTTACT-3′ (SEQ ID NO: 9) and 5′-CATACACCTGGAGCCAGACT-3′ (SEQ ID NO: 10), respectively.

As a result, it was confirmed that the effect of gene inhibition in the visceral fat of the solution and the microstructure was similar (see FIG. 12B). These results mean that the presence or absence of HA and the microstructure fabrication process did not affect the efficacy of the complex.

Lastly, after blood collection, blood clots were formed by incubation at room temperature for 30 minutes and centrifugated at 1500 g and 4° C. for 10 minutes, serum was isolated, and blood TNF-α, IL-6, IL-1β, and MCP-1 were analyzed by ELISA (see FIG. 12C).

As a result, it was confirmed that the effect of reducing inflammatory factors by gene inhibition in animals exhibited a similar tendency in the solution and the microstructure. These results mean that the presence or absence of HA and the microstructure fabrication process did not affect the efficacy of the complex.

### Experimental Example 14: Serum Isolation and Identification of Factors Related to Fat Absorption Mechanism

After collecting blood from the tail vein of C57BL/6 mice that had completed treatment, the blood was incubated at room temperature for 30 minutes to form a blood clot, and centrifuged at 1500 g and 4° C. for 10 minutes. Serum was isolated, and blood TG and FFA were measured using the Triglyceride Assay Kit and the Free Fatty Acid Assay Kit.

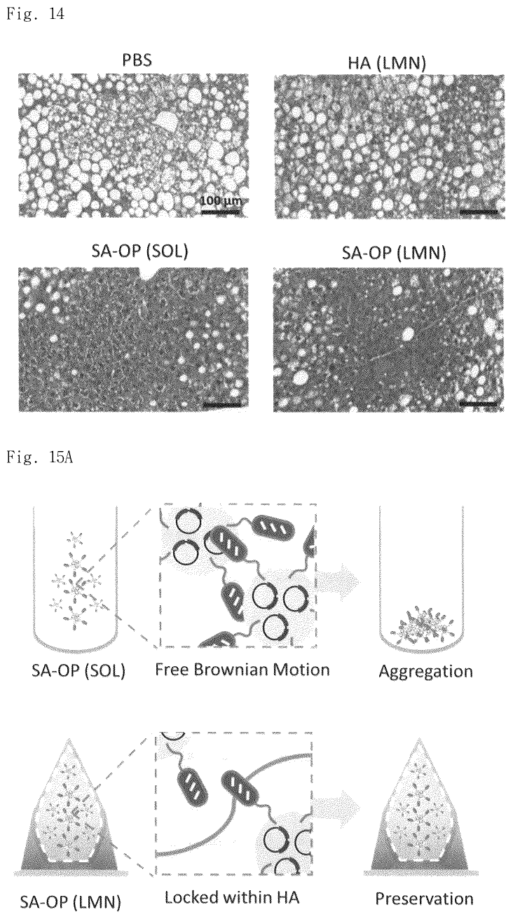

As a result, it was confirmed that the effect of reducing factors related to fatty liver by gene inhibition in animals exhibited a similar tendency in the solution and the microstructure (see FIG. 13). These results mean that the presence or absence of HA and the microstructure fabrication process did not affect the efficacy of the complex.

### Experimental Example 15: Confirmation of Fat Droplet Size in the Liver after Ex Vivo Sampling

The liver tissue of C57BL/6 mice that had completed treatment was fixed in 4% paraformaldehyde, stained by H&E staining, and photographed under a microscope.

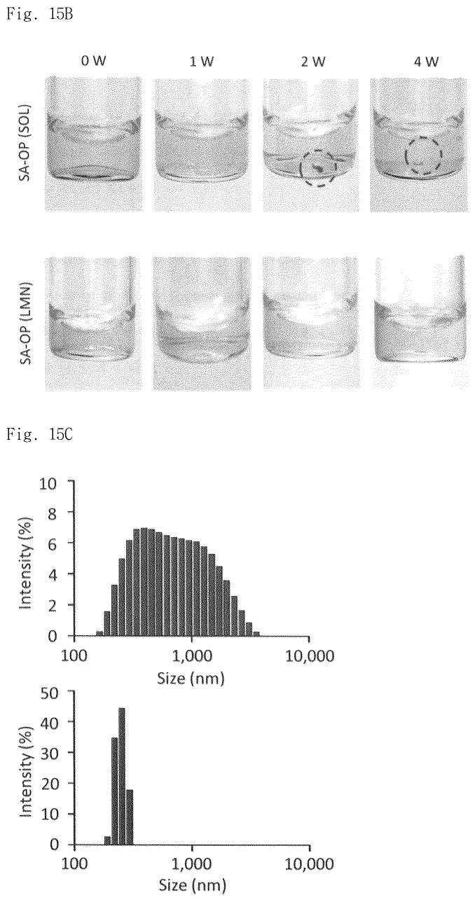

As a result, it was confirmed that the effect of inhibiting fatty liver by gene inhibition in animals exhibited a similar tendency in the solution and the microstructure (see FIG. 14). These results mean that the presence or absence of HA and the microneedle fabrication process did not affect the efficacy of the complex.

### Experimental Example 16: Long-Term Storage Capability of SA-OP-Loaded Microstructure

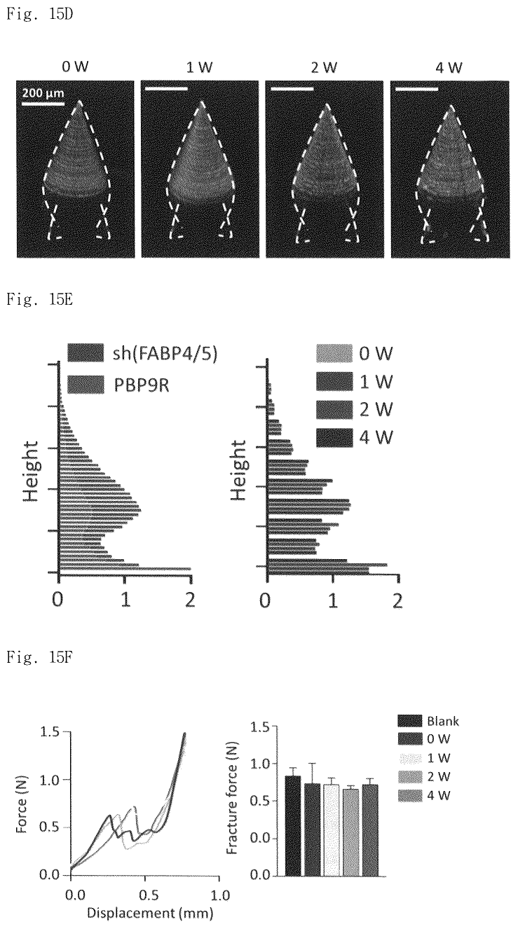

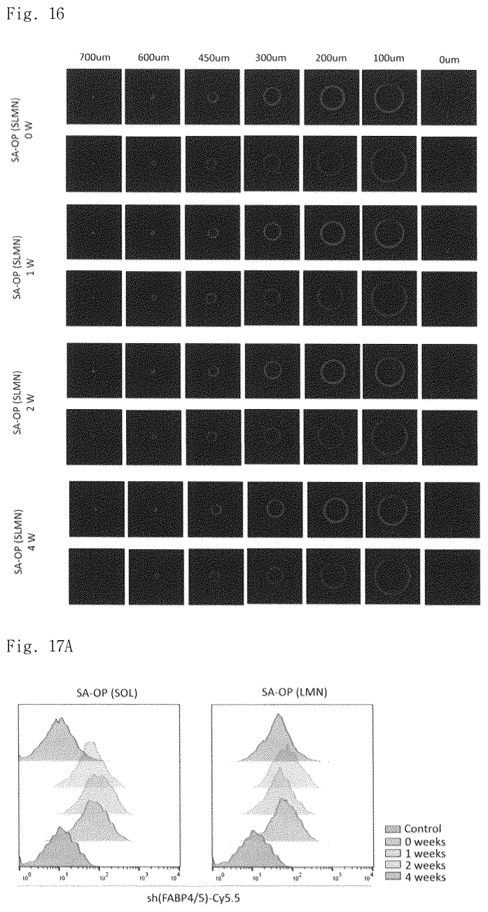

The storage stability of SA-OP-loaded microstructure including a biodegradable HA polymer, which is expected to prevent aggregation by individually separating the complex, was confirmed (see FIG. 15A). Specifically, SA-OP (SOL) and SA-OP (LMN) were stored at 4° C. for four weeks, and their stability was evaluated in various aspects.

To visualize the distribution of SA-OP in the LMN during storage, shRNA was conjugated with Cy5.5 fluorescence, and PBP9R was conjugated to FITC. The aggregation of SA-OP (SOL) was observed after two weeks of storage, and the aggregation was more prominent at week 4 (see FIG. 15B). In addition, to investigate the deformation of SA-OP loaded in the microstructure and SA-OP stored in an aqueous solution, the nanoparticle size within the microstructure after re-dissolution was measured using DLS. The percentage of nanoparticles aggregated from SA-OP (SOL) was observed in the size-distribution graph as mediated electrostatic interactions (see FIG. 15C). However, SA-OP of LMN maintained its original physical properties and exhibited the highest distribution at 200 to 300 nm (see FIG. 15C).

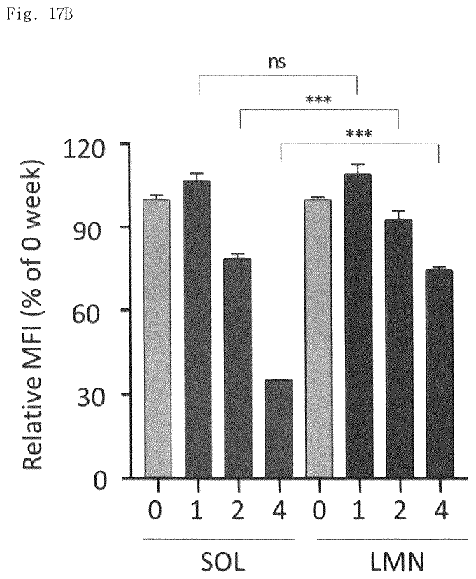

In addition, the distribution of SA-OP in LMN stored for less than four weeks was analyzed using confocal laser microscopy with fluorescence-conjugated SA-OP. The fluorescence-conjugated SA-OP was evenly distributed from the tip to the base of the LMN, and this pattern was maintained over time (see FIGS. 15D, 15E, and 16). The fluorescence intensities of the genes and peptides inside the SA-OP (LMN) were also evenly distributed along the Z-stack of the confocal laser scanning microscopy (CLSM) images, showing the shape of the soluble interlocking microstructures in the graph (see FIG. 15E). In addition, the integrated values of the fluorescence intensities of the genes and peptides stored for four weeks indicate that SA-OP may be captured in the HA backbone of the microneedle and maintain its physicochemical properties (see FIG. 15E). In addition, a mechanical stability evaluation was performed to confirm that LMN stored for a long time can efficiently penetrate the skin to deliver SA-OP (see FIG. 15F). The force-displacement test demonstrated that there was no loss of mechanical stability in the LMN even after four weeks. These results indicate that the soluble LMN can be stored at 4° C. after being fabricated with genetic material without any deformation.

These results suggest that the HA-based backbone of the microstructure prevents SA-OP from interacting with each other and stabilizes the complex until the microstructure is dissolved. In addition, the overall long-term storage capability of the SA-OP-loaded microstructure indicates the possibility of preserving the encapsulated gene/peptide complex for a long period of time, which may provide a significant advantage over storage in a liquid form.

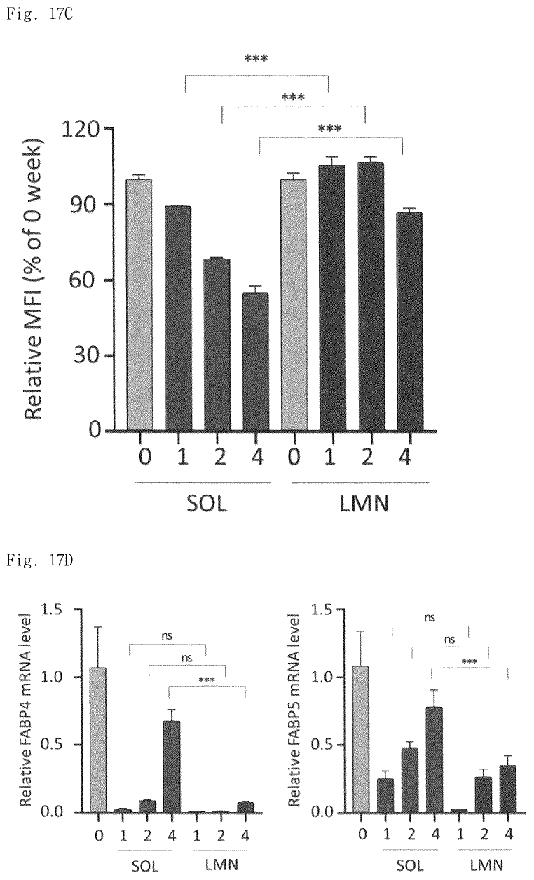

To further investigate whether SA-OP (LMN) stored for a long period of time maintains adipocyte-targeting properties and gene delivery efficacy, flow cytometry was performed using sh(FABP4/5)-Cy5.5 and PBP9R-FITC. Fluorescent-conjugated SA-OP was stored at 4° C. at several time points and then mature 3T3-L1 adipocytes were treated with the SA-OP overnight. In the case of SA-OP (SOL), the mean fluorescence intensity (MFI) of sh(FABP4/5) was significantly reduced by 21.44% and 64.72% at week 2 and week 4 after storage, respectively (see FIGS. 17A and 17B). In contrast, SA-OP (LMN) showed a higher gene delivery efficacy than SA-OP (SOL) with a 39.33% higher MFI value at week 4.

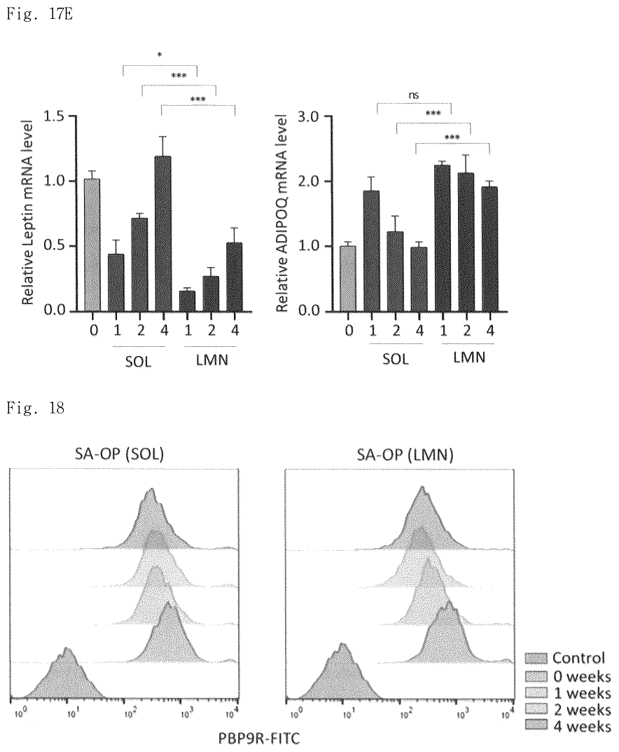

In addition, the MFI value of PBP9R-FITC indicates that SA-OP (LMN) exhibited higher carrier delivery than the corresponding SA-OP (SOL) group (see FIG. 17C and FIG. 18). To confirm the therapeutic effect beyond the gene delivery efficacy of long-term stored SA-OP, mature adipocytes was treated with each sample, and RNA was isolated after 48 hours. A qPCR analysis of the synthesized cDNA showed that SA-OP (LMN) resulted in 60.25% and 42.89% lower FABP4 and FABP5 mRNA levels on the gene silencing targets (FABP4 and FABP5), respectively, compared to SA-OP (SOL) at week 4 (see FIG. 17D).

In addition, leptin, which is an obesity adipocyte biomarker, decreased by 49.18% in the SA-OP (LMN) group at week 4, while no significant difference was observed in the SA-OP (SOL) group (see FIG. 17E). In addition, the mRNA levels of the anti-inflammatory (ADIPOQ) gene increased by 91.04% and 70.97%, respectively, after 4 weeks of storage (see FIG. 17E).

In conclusion, it was confirmed that the soluble interlocking microstructure exhibited long-term stability by preserving the physicochemical properties and therapeutic effects of the genetic material and carrier.

Sequence List Free Text

| | |
|---|---|
| <110> | IUCF-HYU (Industry-University Cooperation Foundation |
| | Hanyang University) |
| <120> | Microstructure comprising a gene and adipocyte-targeted gene |
| | delivery system complex for prevention and treatment of obesity and |
| | obesity-derived type 2 diabetes |
| <210> | 1 |
| <211> | 6046 |
| <212> | DNA |
| <213> | Artificial Sequence |
| <223> | sh(FABP4/5) plasmid |
| <400> | |

cctgcaggcg ttacataact tacggtaaat ggcccgcctg gctgaccgcc caacgacccc
cgcccattga cgtcaataat gacgtatgtt cccatagtaa cgccaatagg gactttccat
tgacgtcaat gggtggagta tttacggtaa actgcccact tggcagtaca tcaagtgtat
catatgccaa gtacgccccc tattgacgtc aatgacggta aatggcccgc ctggcattat
gcccagtaca tgaccttatg ggactttcct acttggcagt acatctacgt attagtcatc
gctattacca tgatgatgcg gttttggcag tacatcaatg ggcgtggata gcggtttgac
tcacggggat ttccaagtct ccaccccatt gacgtcaatg ggagtttgtt ttgactagta
aatcaacggg actttccaaa atgtcgtaac aactccgccc cattgacgca aatgggcggt
aggcgtgtac ggtgggaggt ctatataagc agagctcgtt tagtgaaccg tcagatcagc
ttcgaggggc tcgcatctct ccttcacgcg cccgccgccc tacctgaggc cgccatccac
gccggttgag tcgcgttctg ccgcctcccg cctgtggtgc ctcctgaact gcgtccgccg
tctaggtaag tttaaagctc aggtcgagac cgggcctttg tccggcgctc ccttggagcc
tacctagact cagccggctc tccacgcttt gcctgaccct gcttgctcaa ctctacgtct
ttgtttcgtt ttctgttctg cgccgttaca gatccaagcc accatggttt ctaagggaga
agaactcttt actggtgttg tcccaattct ggttgagctg gatggtgatg tgaatggcca
caaattctct gtgtctggtg aaggtgaagg agatgcaact tatggaaagc tgactctgaa
gttcatttgt acaacaggaa agctgccagt gccttggcca actctggtga ccaccctgac
ttatggtgtt caatgtttca gcagataccc tgaccacatg aagcagcatg acttctttaa
atctgcaatg ccagaaggtt atgttcagga gaggacaatc ttctttaagg atgatggaaa
ttataagaca agggcagaag tgaagtttga aggtgataca ctggttaaca gaattgagct
gaaaggcatt gattttaagg aagatggaaa cattctgggt cacaagctgg agtacaacta
taattctcac aatgtttaca ttatggcaga taagcagagg aatggaatta aggctaattt
caagattaga cacaacattg aggatggatc tgtccaactg gcagaccatt accagcagaa
cacccctatt ggtgatggcc cagttctcct cccagataat cactatctca gcactcaatc
tgctctgtcc aaagacccta atgagaaaag agaccacatg gtcctcctgg agtttgtgac
agcagcagga attactctgg gaatggatga gctgtacaag ggtaagtcac tgactgtcta
tgcctgggaa agggtgggca ggagatgggg cagtgcagga aaagtggcac tatgaaccca
ctagtttgac aattaatcat aagcatagta taatacaact cactatagca attgtactaa
ccttcttctc tttcctctcc tgacaggagg agccatcatg gccaaactca cttctgcagt
cccagtcctc acagcaaggg atgttgcagg ggctgtagag ttctggactg acagattagg
attctccaga gactttgttg aagatgattt tgctggtgtt gtcagagatg atgtcaccct
cttcatctca gcagttcagg accaagttgt ccctgacaac acccttgctt gggtctgggt
cagaggccta gatgagcttt atgcagaatg gtcagaagta gtcagcacaa atttcaggga
tgcctctggc ccagccatga cagaaattgg tgaacaacct tggggaaggg aatttgccct
cagagaccct gctggaaatt gtgtccattt tgtagctgag gaacaggact aaagctagaa
gctcgctttc ttgctgtcca atttctatta aaggttcctt tgttccctaa gtccaactac
taaactgggg gatattatga agggccttga gcatctggat tctgcctaat aaaaaacatt
tattttcatt gcaatgatgt atttaaatta tttctgaata ttttactaaa aagggaatgt
gggaggtcag tgcatttaaa acataaagaa atgaagagct agttcaaacc ttgggaaaat
acactatatc ttaaactcca tgaaagaagg tgaggctgca aacagctaat gcacattggc
aacagcccct gatgcctatg ccttattcat ccctcagaaa aggattcaag tagaggcttg
atttggaggt taaagttttg ctatgctgta ttttaattaa cgttctgcag tatttagcat
gccccaccca tctgcaaggc attctggata gtgtcaaaac agctggaaat caagtctgtt
tatctcaaac tttagcattt tgggaataaa tgatatttgc tatgctggtt aaattagatt
ttagttaaat ttcctgctga agctctagta tgataagtaa cttgacctaa gtgtaaagtt
gagatttcct tcaggtttat atagtcccta tcagtgatag agacctcggt cttcacctga
ggtttttcaa aagtagttga caattaatca tcggcatagt atatcggcat agtataatac
gactcactat aggagggcca ccatggtccg tcctgtagaa accccaaccc gtgaaatcaa
aaaactcgac ggcctgtggg cattcagtct ggatcgcgaa aactgtggaa ttgatcagcg
ttggtgggaa agcgcgttac aagaaagccg ggcaattgct gtgccaggca gttttaacga
tcagttcgcc gatgcagata ttcgtaatta tgcgggcaac gtctggtatc agcgcgaagt
ctttataccg aaaggttggg caggccagcg tatcgtgctg cgtttcgatg cggtcactca
ttacggcaaa gtgtgggtca ataatcagga agtgatggag catcagggcg gctatacgcc
atttgaagcc gatgtcacgc cgtatgttat tgccgggaaa agtgtacgta tcaccgtttg
tgtgaacaac gaactgaact ggcagactat cccgccggga atggtgatta ccgacgaaaa
cggcaagaaa aagcagtctt acttccatga tttctttaac tatgccggaa tccatcgcag
cgtaatgctc tacaccacgc cgaacacctg ggtggacgat atcaccgtgg tgacgcatgt
cgcgcaagac tgtaaccacg cgtctgttga ctggcaggtg gtggccaatg gtgatgtcag
cgttgaactg cgtgatgcgg atcaacaggt ggttgcaact ggacaaggca ctagcgggac
tttgcaagtg gtgaatccgc acctctggca accgggtgaa ggttatctct atgaactgtg
cgtcacagcc aaaagccaga cagagtgtga tatctacccg cttcgcgtcg gcatccggtc
agtggcagtg aagggcgaac agttcctgat taaccacaaa ccgttctact ttactggctt
tggtcgtcat gaagatgcgg acttacgtgg caaaggattc gataacgtgc tgatggtgca
cgaccacgca ttaatggact ggattggggc caactcctac cgtacctcgc attaccctta
cgctgaagag atgctcgact gggcagatga acatggcatc gtggtgattg atgaaactgc
tgctgtcggc tttaacctct ctttaggcat tggtttcgaa gcgggcaaca agccgaaaga
actgtacagc gaagaggcag tcaacgggga aactcagcaa gcgcacttac aggcgattaa
agagctgata gcgcgtgaca aaaaccaccc aagcgtggtg atgtggagta ttgccaacga
accggatacc cgtccgcaag gtgcacggga atatttcgcg ccactggcgg aagcaacgcg
taaactcgac ccgacgcgtc cgatcacctg cgtcaatgta atgttctgcg acgctcacac
cgataccatc agcgatctct ttgatgtgct gtgcctgaac cgttattacg gatggtatgt
ccaaagcggc gatttggaaa cggcagagaa ggtactggaa aaagaacttc tggcctggca
ggagaaactg catcagccga ttatcatcac cgaatacggc gtggatacgt tagccgggct
gcactcaatg tacaccgaca tgtggagtga agagtatcag tgtgcatggc tggatatgta
tcaccgcgtc tttgatcgcg tcagcgccgt cgtcggtgaa caggtatgga atttcgccga
ttttgcgacc tcgcaaggca tattgcgcgt tggcggtaac aagaaaggga tcttcactcg
cgaccgcaaa ccgaagtcgg cggcttttct gctgcaaaaa cgctggactg gcatgaactt
cggtgaaaaa ccgcagcagg gaggcaaaca ataatagcta gaggaagact ttttggaaaa
gattaaaaac ccgcttcggc gggttttttt atgcatgtga gcaaaaggcc agcaaaaggc
caggaaccgt aaaaaggccg cgttgctggc gtttttccat aggctccgcc cccctgacga
gcatcacaaa aatcgacgct caagtcagag gtggcgaaac ccgacaggac tataaagata
ccaggcgttt ccccctggaa gctccctcgt gcgctctcct gttccgaccc tgccgcttac
cggatacctg tccgcctttc tcccttcggg aagcgtggcg ctttctcata gctcacgctg
taggtatctc agttcggtgt aggtcgttcg ctccaagctg ggctgtgtgc acgaaccccc
cgttcagccc gaccgctgcg ccttatccgg taactatcgt cttgagtcca acccggtaag
acacgactta tcgccactgg cagcagccac tggtaacagg attagcagag cgaggtatgt
aggcggtgct acagagttct tgaagtggtg gcctaactac ggctacacta gaagaacagt
atttggtatc tgcgctctgc tgaagccagt taccttcgga aaaagagttg gtagctcttg
atccggcaaa caaaccaccg ctggtagcgg tggttttttt gtttgcaagc agcagattac
gcgcagaaaa aaaggatctc aagaagatcc tttgatcttt tctacggggt ctgacgctca
gtggaacgaa aactcacgtt aagggatttt ggtcatgttc ttaatcgata ctagtgctgc
agtatttagc atgccccacc catctgcaag gcattctgga tagtgtcaaa acagccggaa
atcaagtccg tttatctcaa actttagcat tttgggaata aatgatattt gctatgctgg
ttaaattaga ttttagttaa atttcctgct gaagctctag tacgataagt aacttgacct
aagtgtaaag ttgagatttc cttcaggttt atatagcttg tgcgccgcct gggtacctga
ggtttttcaa aagtagttga caattaatca tcggcatagt atatcggcat agtataatac
gactcactat aggagggcca ccatggaccc tgttgtgctg caaaggagag actgggagaa
ccctggagtg acccagctca acagactggc tgcccaccct ccctttgcct cttggaggaa
ctctgaggaa gccaggacag acaggcccag ccagcagctc aggtctctca atggagagtg
gaggtttgcc tggttccctg cccctgaagc tgtgcctgag tcttggctgg agtgtgacct
cccagaggct gacactgtgt aaccctaagc ttctagactt aattaa

| | |
|---|---|
| <210> | 2 |
| <211> | 18 |
| <212> | PRT |
| <213> | Artificial Sequence |
| <220> | |
| <223> | ATS-9R |
| <400> | 2 |

Cys Lys Gly Gly Arg Ala Lys Asp Arg Arg Arg Arg Arg Arg Arg Arg
Arg Cys

| | |
|---|---|
| <210> | 3 |
| <211> | 22 |
| <212> | DNA |
| <213> | Artificial Sequence |
| <220> | |
| <223> | FABP4 Forward primer |
| <400> | 3 |

tgaaatcacc gcagacgaca gg

| | |
|---|---|
| <210> | 4 |
| <211> | 23 |
| <212> | DNA |
| <213> | Artificial Sequence |
| <220> | |
| <223> | FABP4 Reverse primer |
| <400> | 4 |

gcttgtcacc atctcgtttt ctc

| | |
|---|---|
| <210> | 5 |
| <211> | 21 |
| <212> | DNA |
| <213> | Artificial Sequence |
| <220> | |
| <223> | FABP5 Forward primer |
| <400> | 5 |

tggtttaccc aggatcattc c

| | |
|---|---|
| <210> | 6 |
| <211> | 22 |
| <212> | DNA |
| <213> | Artificial Sequence |
| <220> | |
| <223> | FABP5 Reverse primer |
| <400> | 6 |

cctgaagaat accagagagc tt

| | |
|---|---|
| <210> | 7 |
| <211> | 21 |
| <212> | DNA |
| <213> | Artificial Sequence |
| <220> | |
| <223> | Leptin Forward primer |
| <400> | 7 |

tgagtttgtc caagatggac c

| | |
|---|---|
| <210> | 8 |
| <211> | 18 |
| <212> | DNA |
| <213> | Artificial Sequence |
| <220> | |
| <223> | Leptin Reverse primer |
| <400> | 8 |

gccatccagg ctctctgg

| | |
|---|---|
| <210> | 9 |
| <211> | 22 |
| <212> | DNA |
| <213> | Artificial Sequence |
| <220> | |
| <223> | adiponectin Forward primer |
| <400> | 9 |

caatgtaccc attcgcttta ct

| | |
|---|---|
| <210> | 10 |
| <211> | 20 |
| <212> | DNA |
| <213> | Artificial Sequence |
| <220> | |
| <223> | adiponectin Reverse primer |
| <400> | 10 |

catacacctg gagccagact

1. A microstructure for preventing or treating obesity or obesity-derived type 2 diabetes, comprising a complex including an obesity or obesity-derived type 2 diabetes treatment gene and an adipocyte-targeting carrier.

2. The microstructure of claim 1, wherein the obesity or obesity-derived type 2 diabetes treatment gene targets fatty acid-binding protein 4 (FABP4, aP2) or fatty acid-binding protein 5 (FABP5).

3. The microstructure of claim 1, wherein the adipocyte-targeting carrier is ATS-9R peptide.

4. The microstructure of claim 3, wherein the ATS-9R peptide is represented by SEQ ID NO: 2.

5. The microstructure of claim 1, further comprising a biocompatible polymer.

6. The microstructure of claim 5, wherein the biocompatible polymer is hyaluronic acid.

7. The microstructure of claim 1, wherein the microstructure is dissolved within the skin.

8. A patch for preventing or treating obesity or obesity-derived type 2 diabetes, comprising the microstructure of claim 1.

9. The patch of claim 8, the patch includes a base film and a plurality of microneedles.

10. A method of manufacturing a microstructure for preventing or treating obesity or obesity-derived type 2 diabetes, the method comprising:
(a) a step of preparing an obesity or obesity-derived type 2 diabetes treatment gene;
(b) a step of preparing an adipocyte-targeting carrier; and
(c) a step of manufacturing a microstructure including a complex including the gene of Step (a) and the carrier of Step (b).
